(19) United States
(12) Patent Application Publication
(10) Pub. No.: US 2024/0351996 A1
(43) Pub. Date: Oct. 24, 2024

(54) CDK19-SELECTIVE INHIBITORS, AND METHODS OF USE THEREOF

(71) Applicants: CZ BIOHUB SF, LLC, San Francisco, CA (US); THE BOARD OF TRUSTEES OF THE LELAND STANFORD JUNIOR UNIVERSITY, Stanford, CA (US)

(72) Inventors: Vincent Alford, Stanford, CA (US); Mark Smith, Stanford, CA (US); Michael F. Clarke, Stanford, CA (US); Jitendra Gurjar, Palo Alto, CA (US); Angera Hsiao-Chi Kuo, Foster City, CA (US)

(21) Appl. No.: 18/577,851

(22) PCT Filed: Jul. 11, 2022
(86) PCT No.: PCT/US2022/036692

§ 371 Date: Jan. 9, 2024

(51) Int. Cl. C07D 401/12; A61K 31/444; A61K 31/506; C07D 213/74; C07D 239/42; C07D 239/48; C07D 401/06; C07D 401/14; C07D 405/14

(52) U.S. Cl. CPC C07D 401/12; A61K 31/444; A61K 31/506; C07D 213/74; C07D 239/42; C07D 239/48; C07D 401/06; C07D 401/14; C07D 405/14

(57) ABSTRACT

Provided herein are compounds, tautomers, or pharmaceutically acceptable salts thereof, having a structure of formula (I): wherein X¹, Y, Z¹, Z², (R¹)ₙ, (R²)ₘ, and ring A are as described herein. Also provided are pharmaceutical compositions comprising compounds, tautomers, or pharmaceutically acceptable salts having a structure of formula (I). Further provided are a method of inhibiting cyclin dependent kinase 19 (CDK19) a method of treating breast cancer with the disclosed compounds.

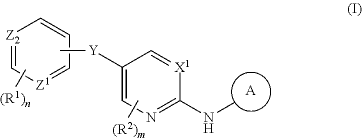

### STATEMENT OF GOVERNMENT SUPPORT

This invention was made with government support under Contract No. BC123235 awarded by the Department of Defense. The government has certain rights in the invention.

### BACKGROUND

Although the survival rate of breast cancer patients has improved over the past 30 years, breast cancer still remains the leading cause of cancer-related death among women worldwide. This decrease in patient mortality rates can be primarily attributed to early cancer detection methods such as routinely administered mammograms and screening of genetic biomarkers associated within high risk patient populations. Unfortunately, scientific advancements in targeted therapeutic strategies have proven more difficult to achieve. For example, target-specific therapies such as tamoxifen and Herceptin display efficacy in more commonly diagnosed breast cancer cases yet have shown to be completely ineffective in treating a subset of patients diagnosed with triple-negative breast cancer (TNBC). TNBC is an aggressive, invasive breast cancer subtype that is characterized as estrogen receptor (ER) negative, progesterone receptor (PR) negative, and HER2-negative, that is, a “triple-negative” phenotype. For this reason, the only therapeutic intervention left available to these patients is chemotherapy, which is known to be non-specific and highly cytotoxic. For example, current strategies for treating TNBC include inhibiting transcriptional co-factors and targeting cancer stem cells, of which both approaches are limited by toxicity. Accordingly, patients diagnosed with TNBC often experience worse survival outcomes than non-TNBCs (median survival 9 months vs 22 months, respectively).

Cyclin dependent kinase 19 (CDK19), and a related isoform CDK8, are oncogenic transcription-regulating kinases that play a role in certain cancers, including TNBC. Other cancers include, but are not limited to, prostate cancer, cancer of the gastrointestinal tract (e.g., colorectal cancer), bladder cancer, sarcoma, cervical cancer, esophageal adenocarcinoma, acute myeloid leukemia, melanoma, glioma, and ovarian cancer. Compounds that non-selectively inhibit CDK19 and CDK8 have been explored for their anti-cancer properties, but have shown to have undesired side effects due to the CDK8 inhibition. Inhibition of CDK8 typically leads to greater side-effects due to its wider tissue distribution as compared to CDK19. For example, compounds that inhibit CDK8 typically result in greater gastrointestinal side-effects owing to the relatively high expression of CDK8 in the colon. It is believed that compounds that selectively inhibit CDK19 would result in a greater therapeutic index and would have less systemic toxicity.

In view of the foregoing, there remains a need for compounds that selectively inhibit CDK19 over CDK8, as well as new methods of treating cancer, such as TNBC, which comprising administering these compounds.

### SUMMARY

The disclosure provides compounds, tautomer, or pharmaceutically acceptable salt thereof, having a structure of formula (I):

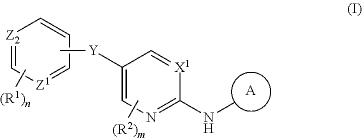

wherein:
X¹ is CH, CR², or N;
Y is selected from the group consisting of a bond, CR^{a}R^{b}, NR^{c}, C(O), O, S, SO₂, C(O)NH, and HNC(O);
each of Z¹ and Z² is independently CH, CR¹, or N;
each of R^{a} and R^{b} is independently H, C₁-C₆alkyl, hydroxy, or halo, or R^{a} and R^{b} taken together with the carbon atom which they are attached form a spiro C₃-C₆cycloalkyl;
R^{c} is H or C₁-C₆alkyl;
ring A comprises a C₆-C₁₀aryl, a C₃-C₆ cycloalkyl, or a 6-membered cycloheteroalkyl comprising 1, 2, or 3 ring heteroatoms independently selected from N, O, and S, wherein ring A is optionally substituted with 1-3 substituents independently selected from the group consisting of halo, hydroxy, —CN, C₁-C₆alkyl, C₁-C₆haloalkyl, C₃-C₆cycloalkyl, a spiro C₃-C₆ cycloalkyl, C₁-C₆alkoxy, C₁-C₆haloalkoxy, C₃-C₆cycloalkoxy, C₃-C₆cycloalkyl-C₁-C₆alkylene, C₆-C₁₀aryl, C₅-C₁₀cycloalkyl, 5-10 membered cycloheteroalkyl comprising 1, 2, or 3 ring heteroatoms independently selected from N, O, and S, NR′R″, and C(O)NR′R″;
n is 0, 1, or 2;
each R¹ is independently selected from the group consisting of halo, hydroxy, cyano, C₁-C₆alkyl, C₁-C₆haloalkyl, C₃-C₆cycloalkyl, C₁-C₆alkoxy, C₁-C₆haloalkoxy, C₂-4alkynylene-phenyl, C₃-C₆cycloalkoxy optionally substituted with C₁-C₆alkyl, C₅-C₆heteroaryl comprising 1, 2, or 3 ring heteroatoms independently selected from N, O, and S NR′R″, C(O)NR′R″, and 6-10 membered cycloheteroalkoxy comprising 1, 2, or 3 ring heteroatoms independently selected from N, O, and S, and the cycloalkyl, cycloalkoxy, phenyl, heteroaryl, and cycloheteroalkoxy ring is substituted with 0, 1, or 2 substituents independently selected from C₁₋₆alkyl, halo, C₁₋₆alkoxy, C₁₋₆haloalkyl, C₁₋₆haloalkoxy, and C₃₋₆cycloalkyl; or
when two R¹ are ortho to each other, taken together with the atoms to which they are attached they form a fused 5 or 6 membered aromatic ring comprising 0-3 ring heteroatoms independently selected from N, O, and S, and is optionally substituted with 1-2 substituents independently selected from C₁-C₆alkyl and oxo;
m is 0, 1, or 2;
each R² is independently C₁-C₆ alkyl;
each R′ and R″ is independently selected from the group consisting of H, C₁-C₁₀alkyl, and C₃-C₆cycloalkyl; or taken together with the nitrogen to which they are attached form a 4-8 membered heterocycle including 0-2 additional ring heteroatoms independently selected from N, O, and S;
with the proviso that when X¹ is N, m is 0, Z¹ is N, Y is para to Z¹, Z² is CH, ring A is phenyl optionally substituted with NH₂ or CH₃, n is 0 or 2, and each R¹ is NH₂, then Y is not a bond.

The disclosure also provides compounds or salts having a structure of formula (IA)-(IG):

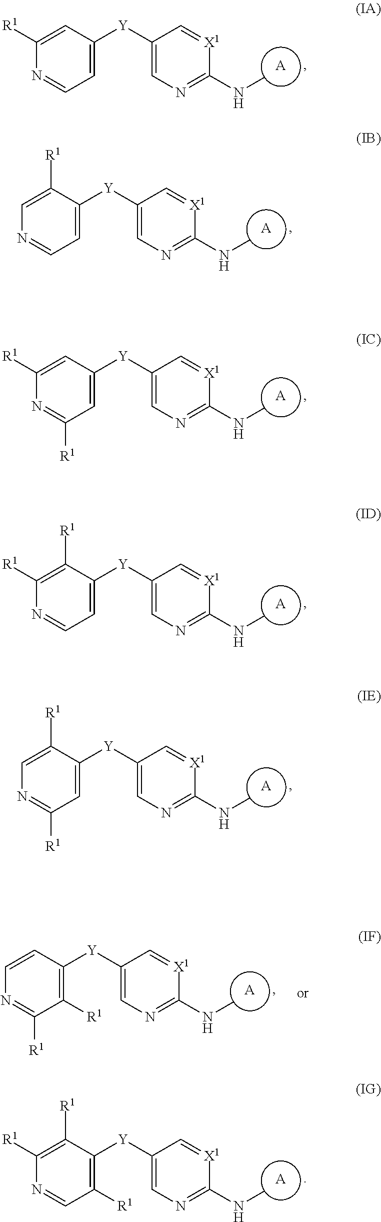

The disclosure also provides pharmaceutical compositions comprising a compound, tautomer, or pharmaceutically acceptable salt thereof, as disclosed herein, and methods of using the disclosed compounds, such as methods of inhibiting CDK19, and methods of treating breast cancer (e.g., triple negative breast cancer).

### BRIEF DESCRIPTION OF THE FIGURES

FIG. 1A shows lethal dose studies of Compound A11 in TNBC and normal fibroblast cells.

FIG. 1B shows lethal dose studies of Compound A53 in TNBC and normal fibroblast cells.

### DETAILED DESCRIPTION

The compounds disclosed herein are inhibitors of CDK19. Inhibition of CDK19 has been shown to be effective against breast cancer, such as triple negative breast cancer. In some embodiments, the disclosed compounds inhibit CDK19 selectively over CDK8, which is a structurally similar CDK but is much more prevalent throughout the body and can lead to many undesired effects, due to its wider tissue distribution as compared to CDK19. In particular, CDK8 inhibition has been shown to have high incidences of gastrointestinal side effects due to the high levels of CDK8 in the colon.

The disclosed compounds bind to and inhibit the activity of CDK19. In some embodiments, the disclosed compounds selectively inhibit CDK19 over CDK8. The compounds disclosed herein can selectively inhibit CDK19 over the isoform CDK8 such that such side effects due to CDK8 inhibition are minimized or avoided, compared to other CDK19 inhibitors.

### Compounds of the Disclosure

Provided herein are compounds, tautomers, or pharmaceutically acceptable salts thereof, having a structure of formula (I):

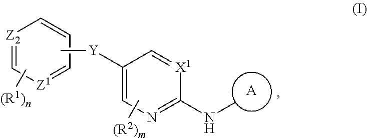

wherein:
X¹ is CH, CR², or N;
Y is selected from the group consisting of a bond, CR^{a}R^{b}, NRC, C(O), O, S, SO₂, C(O)NH, and HNC(O);
each of Z¹ and Z² is independently CH, CR¹, or N;
each of R^{a} and R^{b} is independently H, C₁-C₆alkyl, hydroxy, or halo, or R^{a} and R^{b} taken together with the carbon atom which they are attached form a spiro C₃-C₆cycloalkyl;
R^{c} is H or C₁-C₆alkyl;
ring A comprises a C₆-C₁₀aryl, a C₃-C₆ cycloalkyl, or a 6-membered cycloheteroalkyl comprising 1, 2, or 3 ring heteroatoms independently selected from N, O, and S, wherein ring A is optionally substituted with 1-3 substituents independently selected from the group consisting of halo, hydroxy, —CN, C₁-C₆alkyl, C₁-C₆haloalkyl, C₃-C₆cycloalkyl, a spiro C₃-C₆ cycloalkyl, C₁-C₆alkoxy, C₁-C₆haloalkoxy, C₃-C₆cycloalkoxy, C₃-C₆cycloalkyl-C₁-C₆alkylene, C₆-C₁₀aryl, C₅-C₁₀cycloalkyl, 5-10 membered cycloheteroalkyl comprising 1, 2, or 3 ring heteroatoms independently selected from N, O, and S, NR′R″, and C(O)NR′R″;
n is 0, 1, or 2;
each R¹ is independently selected from the group consisting of halo, hydroxy, cyano, C₁-C₆alkyl, C₁-C₆haloalkyl, C₃-C₆cycloalkyl, C₁-C₆alkoxy, C₁-C₆haloalkoxy, C₂-4alkynylene-phenyl, C₃-C₆cycloalkoxy optionally substituted with C₁-C₆alkyl, C₅-C₆heteroaryl comprising 1, 2, or 3 ring heteroatoms independently selected from N, O, and S NR′R″, C(O)NR′R″, and 6-10 membered cycloheteroalkoxy comprising 1, 2, or 3 ring heteroatoms independently selected from N, O, and S, and the cycloalkyl, cycloalkoxy, phenyl, heteroaryl, and cycloheteroalkoxy ring is substituted with 0, 1, or 2 substituents independently selected from C₁₋₆alkyl, halo, C₁₋₆alkoxy, C₁-C₆haloalkyl, C₁₋₆haloalkoxy, and C₃₋₆cycloalkyl; or
when two R¹ are ortho to each other, taken together with the atoms to which they are attached they form a fused 5 or 6 membered aromatic ring comprising 0-3 ring heteroatoms independently selected from N, O, and S, and is optionally substituted with 1-2 substituents independently selected from C₁-C₆alkyl and oxo;
m is 0, 1, or 2;
each R² is independently C₁-C₆ alkyl;
each R′ and R″ is independently selected from the group consisting of H, C₁-C₁₀alkyl, and C₃-C₆cycloalkyl; or taken together with the nitrogen to which they are attached form a 4-8 membered heterocycle including 0-2 additional ring heteroatoms independently selected from N, O, and S;
   with the proviso that when X¹ is N, m is 0, Z¹ is N, Y is para to Z¹, Z² is CH, ring A is phenyl optionally substituted with NH₂ or CH₃, n is 0 or 2, and each R¹ is NH₂, then Y is not a bond.

In some embodiments, the compounds, tautomer, or salts of formula (I) have a structure of formula (IA)-(IG):

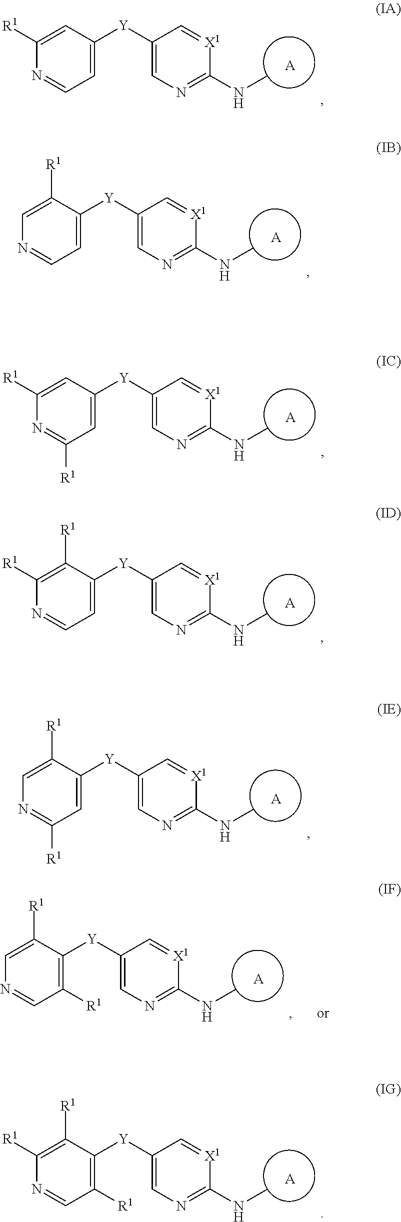

In some embodiments, the compounds, tautomers, or salts of formula (I) have a structure of formula (IA).

The compounds disclosed herein include all pharmaceutically acceptable isotopically-labeled compounds wherein one or more atoms of the compounds disclosed herein are replaced by atoms having the same atomic number, but an atomic mass or mass number different from the atomic mass or mass number usually found in nature, examples of which include isotopes of hydrogen, such as ²H and ³H. In some cases, one or more hydrogen atoms of the compounds disclosed herein are specifically deuterium (²H).

It is understood that, in any compound disclosed herein having one or more chiral centers, if an absolute stereochemistry is not expressly indicated, then each center may independently be of (R)-configuration or(s)-configuration or a mixture thereof. Thus, the compounds provided herein may be enantiomerically pure or be stereoisomeric mixtures. Further, compounds provided herein may be racemic mixtures. In addition, it is understood that in any compound having one or more double bond(s) generating geometrical isomers that can be defined as (E) or (z) each double bond may independently be (E) or (z) or a mixture thereof. Likewise, all tautomeric forms are also intended to be included.

The term “alkyl” as used herein refers to a saturated straight or branched chain hydrocarbon. The term “cycloalkyl” refers to a non-aromatic carbon only containing ring system which is saturated, having three to six ring carbon atoms. Examples of C₁-C₆ alkyl groups include but are not limited to methyl, ethyl, isopropyl, n-propyl, isobutyl, n-butyl, sec-butyl, tert-butyl, isopentyl, n-pentyl, neopentyl, sec-pentyl, 3-pentyl, sec-isopentyl, active pentyl, isohexyl, n-hexyl, sec-hexyl, neohexyl, and tert-hexyl. Contemplated C₃-C₆ cycloalkyl groups include cyclopropyl, cyclobutyl, cyclopentyl, and cyclohexyl. An alkylene group is an alkyl group that is further substituted. For example, “alkylene-cycloalkyl” refers to an alkyl group substituted with a cycloalkyl group.

The term “alkynyl” as used herein refers to an unsaturated alkyl group comprising a triple bond. Suitable nonlimiting alkynyl groups include C₂-C₄alkynyl groups, including for example, ethynyl, 1-propynyl, 2-butynyl. An “alkynylene” is an alkynyl group that is further substituted—e.g., alkynylene-phenyl.

The term “haloalkyl” refers to an alkyl substituted with one or more halogen atoms. This term includes perfluorinated alkyl groups, such as —CF₃ and —CF₂CF₃.

The term “alkoxy” refers to an —O-alkyl group wherein the moiety is attached through an oxygen atom. The term “cycloalkoxy” refers to an —O-cycloalkyl group wherein the moiety is attached through an oxygen atom.

The term “haloalkoxy” refers to an alkoxy group substituted with one or more halogen atoms. This term includes perfluorinated alkoxy groups, such as —OCF₃ and —OCF₂CF₃.

As used herein, the term “cyano” refers to —CN.

As used herein, the term “aryl” refers to a monocyclic or bicyclic aromatic group having 6 to 10 ring carbons. Aryl groups can be isolated (e.g., phenyl) or fused to another aryl group (e.g., naphthyl), or a cycloalkyl group (e.g. tetraydronaphthyl). The aryl ring can be substituted as disclosed herein or unsubstituted.

The term “heteroaryl” refers to an “aryl” group as described herein, wherein the ring(s) comprise 1, 2, or 3 ring heteroatoms independently selected from N, O, and S. Examples of heteroaryl groups include, but are not limited to, imidazolyl, pyridinyl, pyrimidinyl, thiazolyl, triazoyl, oxazolyl, pyrrolyl, and isoxazoyl.

The term “cycloheteroalkyl”, refers to a ring comprising 3 to 10 (e.g., 3, 4, 5, 6, 7, 8, 9, or 10) members of which 1 to 4 (e.g., 1, 2, 3, or 4) ring atoms are heteroatoms selected from N, O, and S, including monocyclic heteroalkyl rings and polycyclic ring systems. Examples of cycloheteroalkyl groups include, but are not limited to, piperidinyl and tetrahydropyranyl.

The term “cycloheteroalkoxy” refers to a cycloheteroalkyl group, as described herein, wherein the moiety is attached through an oxygen atom, e.g., —O-cycloheteroalkyl.

As used herein, the term “spiro” refers to a compound having two rings with one atom common to both rings. For example, a spiro cyclopropyl group has the structure

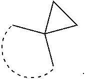

Z¹ and Z²

In some embodiments, each of Z¹ and Z² is independently CH or N. In some embodiments, Z¹ and/or Z² is CR². In some embodiments, Z¹ and Z² are each CH or each are CR². In other embodiments, Z¹ and Z² are each N. In yet other embodiments, Z¹ is N and Z² is CH. In some embodiments, Z¹ is N and Z² is CH. In some embodiments, Z¹ is N and Z² is CR². In some embodiments, Z¹ is CR² and Z² is N.

R¹, R², R′ and R″

In some embodiments, the compounds of the disclosure are substituted with substituents R¹ and R², as described herein, wherein the number of each R¹ and R² is denoted with n and m, respectively (e.g., (R¹), and (R²) m), wherein n is independently 0, 1, 2, or 3 and m is independently 0, 1, or 2. In some embodiments, n is 0, 1, or 2 and m is independently 0, 1, or 2. Thus, the number of R¹ and R² are present in any suitable combination. In some embodiments, n is 0 such that the ring comprising Z¹ and Z² not substituted with R¹. In some embodiments, m is 0 such that the ring comprising X¹ is not substituted with R².

Each R¹ is independently selected from the group consisting of halo, hydroxy, cyano, C₁-C₆alkyl, C₁-C₆haloalkyl, C₃-C₆cycloalkyl, C₁-C₆alkoxy, C₁-C₆haloalkoxy, C₂-4alkynylene-phenyl, C₃-C₆cycloalkoxy optionally substituted with C₁-C₆alkyl, C₅-C₆heteroaryl comprising 1, 2, or 3 ring heteroatoms independently selected from N, O, and S NR′R″, C(O)NR′R″, and 6-10 membered cycloheteroalkoxy comprising 1, 2, or 3 ring heteroatoms independently selected from N, O, and S, and the cycloalkyl, cycloalkoxy, phenyl, heteroaryl, and cycloheteroalkoxy ring is substituted with 0, 1, or 2 substituents independently selected from C₁₋₆alkyl, halo, C₁₋₆alkoxy, C₁₋₆haloalkyl, C₁₋₆haloalkoxy, and C₃₋₆cycloalkyl. In some embodiments, each R¹ is independently selected from the group consisting of halo, hydroxy, cyano, C₁-C₆alkyl, C₁-C₆haloalkyl, C₃-C₆cycloalkyl, C₁-C₆alkoxy, C₁-C₆haloalkoxy, C₃-C₆cycloalkoxy, NR′R″, C(O)NR′R″, and 6-10 membered cycloheteroalkoxy comprising 1, 2, or 3 ring heteroatoms independently selected from N, O, and S; or when two R¹ are ortho to each other, taken together with the atoms to which they are attached they form a fused 5 or 6 membered aromatic ring comprising 0-3 ring heteroatoms independently selected from N, O, and S, and is optionally substituted with 1-2 substituents selected from C₁-C₆alkyl.

In some embodiments, in conjunction with other above and below embodiments, each R¹ is selected from the group consisting of halo, C₁-C₆alkyl, C₁-C₆alkoxy, C₁-C₆haloalkoxy, C₃-C₆cycloalkyl, C₃-C₆cycloalkoxy, and C(O)NR′R″.

In some embodiments, each R¹ is independently selected from the group consisting H, F, Cl, Br, I, methyl, ethyl, isopropyl, cyclopropyl, butyl, cyclobutyl, pentyl, cyclopentyl, cyclohexyl, hydroxyl, methoxy, fluoromethoxy, difluoromethoxy, trifluoromethoxy, ethoxy, propoxy, isopropoxy, butoxy, cyclobutoxy, pentoxy, cyclopentoxy, hexoxy, cyclohexoxy, pyridinyl, 1-naphthyl, 2-naphthyl, —C(O)NR′R″, methylNH—, ethylNH—, isopropylNH—, cyclopropylNH—, butylNH—, cyclobutylNH—, pentylNH—, cyclopentylNH—, hexylNH—, cyclohexylNH—, heptylNH—, and 6-10 membered cycloheteroalkoxy.

In some embodiments, in conjunction with other above and below embodiments, R¹ is C₁-C₆alkyl (e.g., methyl, ethyl, propyl, butyl, pentyl, hexyl). In some embodiments, R¹ is methyl. In some embodiments, R¹ is C₃-C₆cycloalkoxy (e.g., cyclopropoxy, cyclobutoxy, cyclopentoxy, or cyclohexoxy). In some embodiments, R¹ is cyclopentoxy.

In some embodiments, two R¹ are ortho to each other and taken together with the atoms to which they are attached form a 6 membered aryl, which is optionally substituted. For example, in some embodiments, n is 3 wherein two R¹ are taken together to form a 6-membered aryl (e.g., a fused benzo ring), and the third R¹ is C₁-C₆ alkyl (e.g., methyl).

In some embodiments, R¹ is-C(O)NR′R″, as described herein. In some embodiments, in conjunction with other above and below embodiments, R′ is H. In some embodiments, in conjunction with other above and below embodiments, R″ is C₁-C₆alkyl (e.g., methyl).

### X¹ and Y

In some embodiments, in conjunction with other above and below embodiments, X¹ is N and m is 0. In some embodiments, in conjunction with other above and below embodiments, X¹ is N, m is 1, and R² is methyl. In some embodiments, each of X¹ is CH and m is 0.

In some embodiments, in conjunction with other above and below embodiments, Y is NH. In other embodiments, Y is a bond. In yet other embodiments, Y is NHC(O) or C(O)NH. In still yet other embodiments, Y is CH₂. In some embodiments, Y is C(O).

### Ring A

The disclosed compounds comprise ring A comprising a C₆-C₁₀aryl, a C₃-C₆ cycloalkyl, or a 6-membered cycloheteroalkyl comprising 1, 2, or 3 ring heteroatoms independently selected from N, O, and S, wherein ring A is optionally substituted with 1-3 substituents independently selected from the group consisting of halo, hydroxy, —CN, C₁-C₆alkyl, C₁-C₆haloalkyl, C₃-C₆cycloalkyl, a spiro C₃-C₆ cycloalkyl, C₁-C₆alkoxy, C₁-C₆haloalkoxy, C₃-C₆cycloalkoxy, C₃-C₆cycloalkyl-C₁-C₆alkylene, C₆-C₁₀aryl, C₅-C₁₀cycloalkyl, 5-10 membered cycloheteroalkyl comprising 1, 2, or 3 ring heteroatoms independently selected from N, O, and S, NR′R″, and C(O)NR′R″, as described herein.

In some embodiments, in conjunction with other above and below embodiments, ring A is optionally substituted with 1, 2, or 3 substituents selected from the group consisting of F, Cl, Br, I, hydroxy, NH₂, NHR′, methyl, ethyl, propyl, cyclopropyl, butyl, cyclobutyl, isobutyl, tert-butyl, pentyl, cyclopentyl, hexyl, cyclohexyl, methoxy, fluoromethoxy, difluoromethoxy, trifluoromethoxy, ethoxy, 1,1,2,2-tetrafluoroethoxy, perfluoroethoxy, propoxy, isopropoxy, cyclopropoxy, butoxy, cyclobutoxy, isobutoxy, tert-butoxy, pentoxy, cyclopentoxy, hexoxy, cyclohexoxy, cyclopropylmethyl, cyclobutylmethyl, piperazinyl, morpholinyl, 1-naphthyl, 2-naphthyl, tetrahydronapthyl, and isocromenyl.

In some embodiments, ring A is selected from the group consisting of phenyl, cyclohexyl, 4-piperidinyl, and tetrahydropyranyl, wherein ring A is optionally substituted. In some embodiments, ring A is cyclohexyl optionally substituted. In some embodiments, ring A is cyclohexyl substituted at the 4-position with a substitutent selected from the group consisting of methyl, methoxy, and isopropoxy.

In some embodiments, ring A is 4-piperidinyl optionally substituted. In some embodiments, ring A is 4-piperidinyl substituted on ring N with methyl or isobutyl.

In some embodiments, ring A is tetrahydropyranyl optionally substituted.

In some embodiments, ring A is phenyl optionally substituted. In some embodiments, in conjunction with other above and below embodiments, ring A is phenyl substituted at the 2-position with a substituent selected from the group consisting of F, Cl, Br, I, C₁-C₆alkoxy, hydroxy, NH₂, and NHR′. In some embodiments, in conjunction with other above and below embodiments, ring A is phenyl substituted at the 3-position or 4-position with a substituent selected from the group consisting of F, Cl, Br, I, C₁-C₆alkyl, C₁-C₆alkoxy, C₁-C₆-haloalkyl, C₃-C₆-cycloalkyl, C₃-C₆cycloalkoxy, C₃-C₆cycloalkyl-C₁-C₆alkylene, C₆-C₁₀aryl, C₆-C₁₀cycloalkyl, and 6-10 membered heterocycloalkyl comprising 1, 2, or 3 ring heteroatoms independently selected from N, O, and S. In some embodiments, ring A is phenyl substituted at the 4-position with a substituent selected from the group consisting of F, Cl, Br, I, C₁-C₆alkoxy, hydroxy, NH₂, and NHR′.

In some embodiments, ring A is 4-isobutylphenyl. In some embodiments, ring A is 4-cyclobutylphenyl.

In some embodiments, ring A is

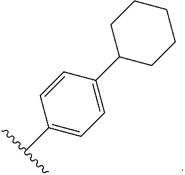 [SMILES: *c1ccc(C2CCCCC2)cc1]

In some embodiments, ring A is

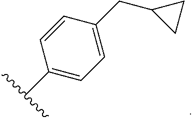 [SMILES: *c1ccc(CC2CC2)cc1]

In some embodiments, ring A is

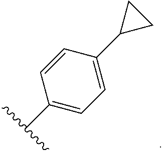 [SMILES: *c1ccc(C2CC2)cc1]

In some embodiments, ring A is

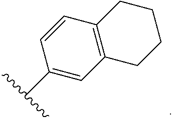 [SMILES: *c1ccc2c(c1)CCCC2]

In some embodiments, ring A is

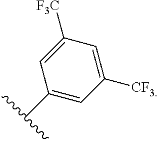 [SMILES: *c1cc(C(F)(F)F)cc(C(F)(F)F)c1]

In some embodiments, ring A is

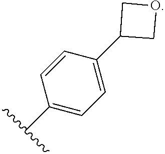 [SMILES: *c1ccc(C2COC2)cc1]

In some embodiments, ring A is

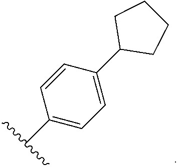 [SMILES: *c1ccc(C2CCCC2)cc1]

In some embodiments, ring A is

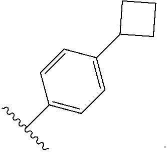 [SMILES: *c1ccc(C2CCC2)cc1]

In some embodiments, ring A is

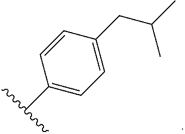 [SMILES: *c1ccc(CC(C)C)cc1]

In some embodiments, ring A is

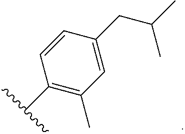 [SMILES: *c1ccc(CC(C)C)cc1C]

In some embodiments, ring A is

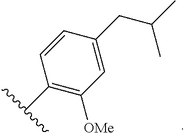 [SMILES: *c1ccc(CC(C)C)cc1OC]

In some embodiments, ring A is

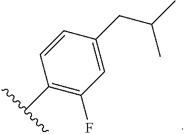 [SMILES: *c1ccc(CC(C)C)cc1F]

In some embodiments, ring A is

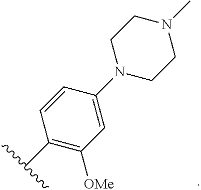 [SMILES: *c1ccc(N2CCN(C)CC2)cc1OC]

In some embodiments, ring A is

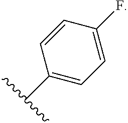 [SMILES: *c1ccc(F)cc1]

In some embodiments, ring A is

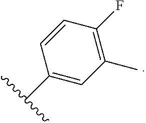 [SMILES: *c1ccc(F)c(C)c1]

In some embodiments, ring A is

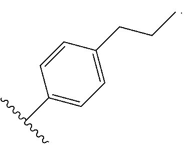 [SMILES: *c1ccc(CCC)cc1]

In some embodiments, ring A is

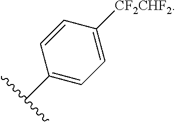

In some embodiments, ring A is

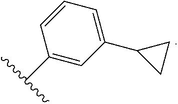 [SMILES: *c1cccc(C2CC2)c1]

In some embodiments, ring A is

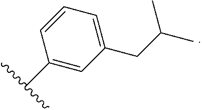 [SMILES: *c1cccc(CC(C)C)c1]

In some embodiments, ring A is

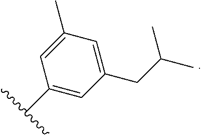 [SMILES: *c1cc(C)cc(CC(C)C)c1]

In some embodiments, ring A is

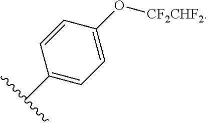

In some embodiments, ring A is

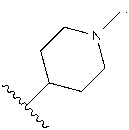 [SMILES: *C1CCN(C)CC1]

In some embodiments, ring A is

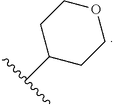 [SMILES: *C1CCOCC1]

In some embodiments, ring A is

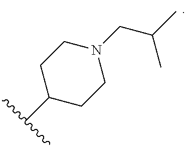 [SMILES: *C1CCN(CC(C)C)CC1]

In some embodiments, ring A is

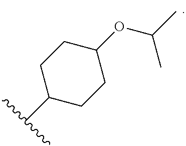 [SMILES: *C1CCC(OC(C)C)CC1]

In some embodiments, ring A is

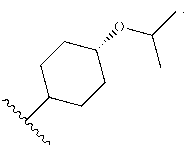

In some embodiments, ring A is

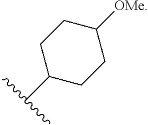 [SMILES: *C1CCC(OC)CC1]

In some embodiments, ring A is

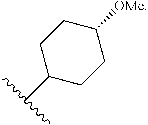

In some embodiments, ring A is

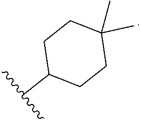 [SMILES: *C1CCC(C)(C)CC1]

In some embodiments, ring A is

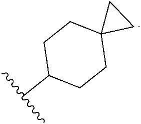 [SMILES: *C1CCC2(CC1)CC2]

In some embodiments, the compound of formula (I) is shown in Table A below, or a pharmaceutically acceptable salt thereof.

TABLE A

| ID | STRUCTURE |
|---|---|
| A1 | 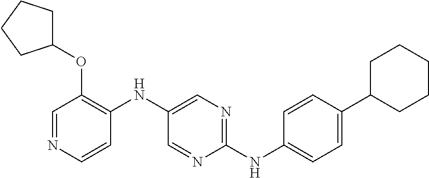 [SMILES: c1cc(Nc2cnc(Nc3ccc(C4CCCCC4)cc3)nc2)c(OC2CCCC2)cn1] |
| A2 | 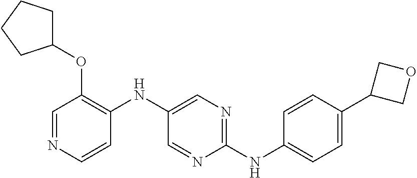 [SMILES: c1cc(Nc2cnc(Nc3ccc(C4COC4)cc3)nc2)c(OC2CCCC2)cn1] |
| A3 | 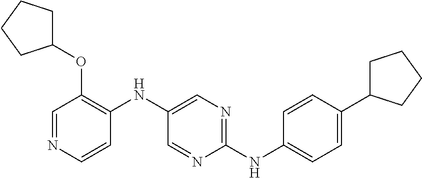 [SMILES: c1cc(Nc2cnc(Nc3ccc(C4CCCC4)cc3)nc2)c(OC2CCCC2)cn1] |
| A4 | 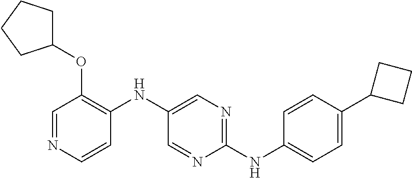 [SMILES: c1cc(Nc2cnc(Nc3ccc(C4CCC4)cc3)nc2)c(OC2CCCC2)cn1] |
| A5 | 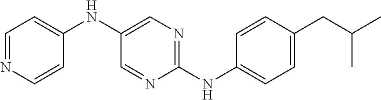 [SMILES: CC(C)Cc1ccc(Nc2ncc(Nc3ccncc3)cn2)cc1] |
| A6 | 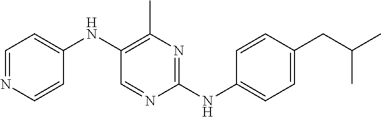 [SMILES: Cc1nc(Nc2ccc(CC(C)C)cc2)ncc1Nc1ccncc1] |
| A7 | 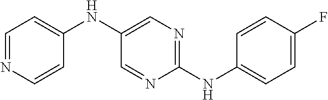 [SMILES: Fc1ccc(Nc2ncc(Nc3ccncc3)cn2)cc1] |
| A8 | 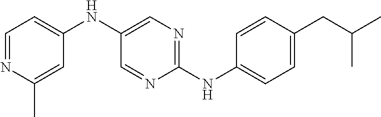 [SMILES: Cc1cc(Nc2cnc(Nc3ccc(CC(C)C)cc3)nc2)ccn1] |
| A9 | 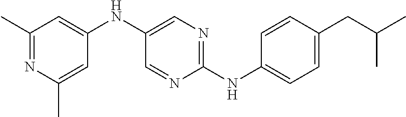 [SMILES: Cc1cc(Nc2cnc(Nc3ccc(CC(C)C)cc3)nc2)cc(C)n1] |
| A10 | 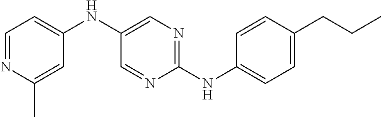 [SMILES: CCCc1ccc(Nc2ncc(Nc3ccnc(C)c3)cn2)cc1] |
| A11 | 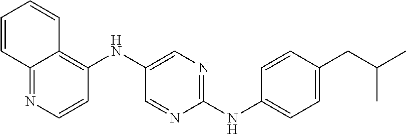 [SMILES: CC(C)Cc1ccc(Nc2ncc(Nc3ccnc4ccccc34)cn2)cc1] |
| A12 | 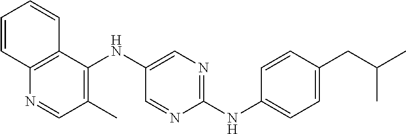 [SMILES: Cc1cnc2ccccc2c1Nc1cnc(Nc2ccc(CC(C)C)cc2)nc1] |
| A13 | 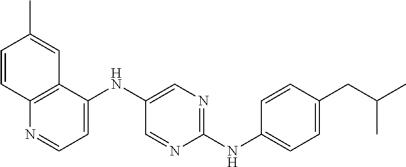 [SMILES: Cc1ccc2nccc(Nc3cnc(Nc4ccc(CC(C)C)cc4)nc3)c2c1] |
| A14 | 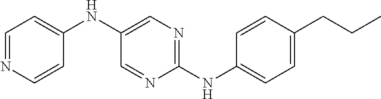 [SMILES: CCCc1ccc(Nc2ncc(Nc3ccncc3)cn2)cc1] |
| A15 | 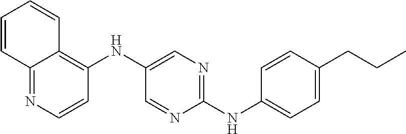 [SMILES: CCCc1ccc(Nc2ncc(Nc3ccnc4ccccc34)cn2)cc1] |
| A16 | 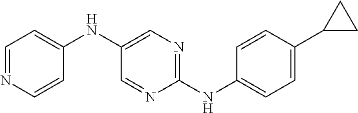 [SMILES: c1cc(Nc2cnc(Nc3ccc(C4CC4)cc3)nc2)ccn1] |
| A17 | 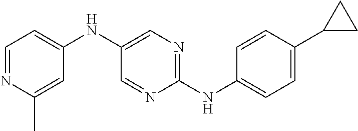 [SMILES: Cc1cc(Nc2cnc(Nc3ccc(C4CC4)cc3)nc2)ccn1] |
| A18 | 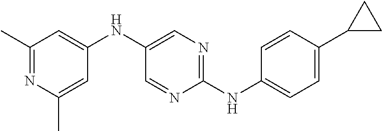 [SMILES: Cc1cc(Nc2cnc(Nc3ccc(C4CC4)cc3)nc2)cc(C)n1] |
| A19 | 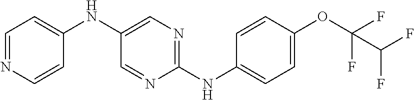 [SMILES: FC(F)C(F)(F)Oc1ccc(Nc2ncc(Nc3ccncc3)cn2)cc1] |
| A20 | 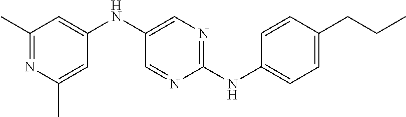 [SMILES: CCCc1ccc(Nc2ncc(Nc3cc(C)nc(C)c3)cn2)cc1] |
| A21 | 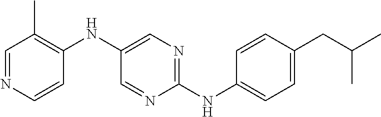 [SMILES: Cc1cnccc1Nc1cnc(Nc2ccc(CC(C)C)cc2)nc1] |
| A22 | 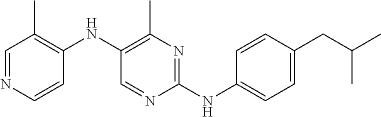 [SMILES: Cc1cnccc1Nc1cnc(Nc2ccc(CC(C)C)cc2)nc1C] |
| A23 | 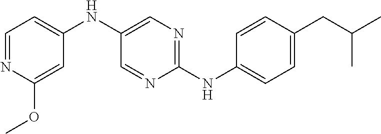 [SMILES: COc1cc(Nc2cnc(Nc3ccc(CC(C)C)cc3)nc2)ccn1] |
| A24 | 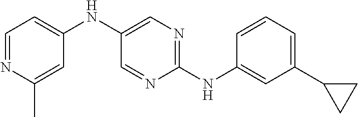 [SMILES: Cc1cc(Nc2cnc(Nc3cccc(C4CC4)c3)nc2)ccn1] |
| A25 | 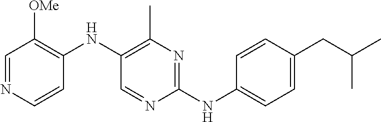 [SMILES: COc1cnccc1Nc1cnc(Nc2ccc(CC(C)C)cc2)nc1C] |
| A26 | 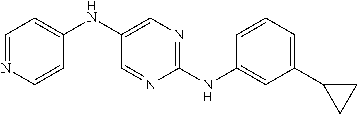 [SMILES: c1cc(Nc2ncc(Nc3ccncc3)cn2)cc(C2CC2)c1] |
| A27 | 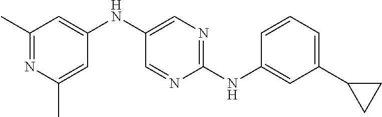 [SMILES: Cc1cc(Nc2cnc(Nc3cccc(C4CC4)c3)nc2)cc(C)n1] |
| A28 | 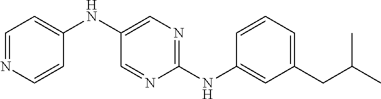 [SMILES: CC(C)Cc1cccc(Nc2ncc(Nc3ccncc3)cn2)c1] |
| A29 | 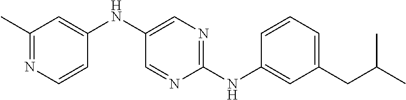 [SMILES: Cc1cc(Nc2cnc(Nc3cccc(CC(C)C)c3)nc2)ccn1] |
| A30 | 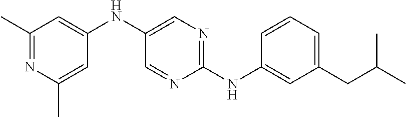 [SMILES: Cc1cc(Nc2cnc(Nc3cccc(CC(C)C)c3)nc2)cc(C)n1] |
| A31 | 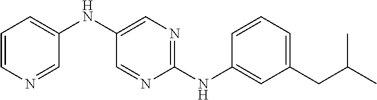 [SMILES: CC(C)Cc1cccc(Nc2ncc(Nc3cccnc3)cn2)c1] |
| A32 | 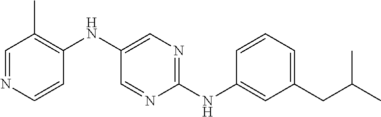 [SMILES: Cc1cnccc1Nc1cnc(Nc2cccc(CC(C)C)c2)nc1] |
| A33 | 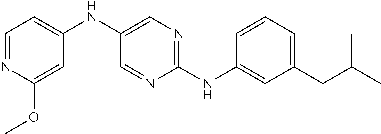 [SMILES: COc1cc(Nc2cnc(Nc3cccc(CC(C)C)c3)nc2)ccn1] |
| A34 | 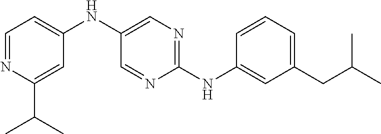 [SMILES: CC(C)Cc1cccc(Nc2ncc(Nc3ccnc(C(C)C)c3)cn2)c1] |
| A35 | 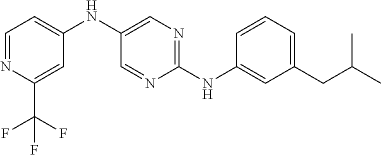 [SMILES: CC(C)Cc1cccc(Nc2ncc(Nc3ccnc(C(F)(F)F)c3)cn2)c1] |
| A36 | 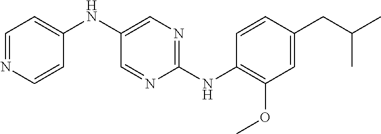 [SMILES: COc1cc(CC(C)C)ccc1Nc1ncc(Nc2ccncc2)cn1] |
| A37 | 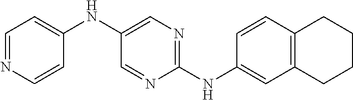 [SMILES: c1cc(Nc2cnc(Nc3ccc4c(c3)CCCC4)nc2)ccn1] |
| A38 | 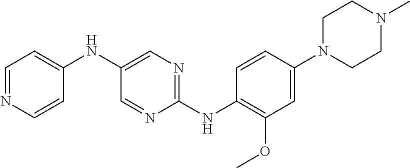 [SMILES: COc1cc(N2CCN(C)CC2)ccc1Nc1ncc(Nc2ccncc2)cn1] |
| A39 | 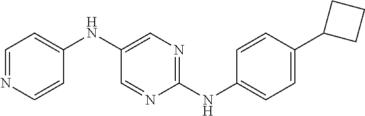 [SMILES: c1cc(Nc2cnc(Nc3ccc(C4CCC4)cc3)nc2)ccn1] |
| A40 | 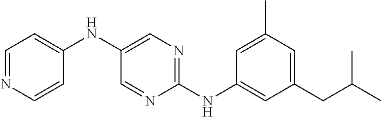 [SMILES: Cc1cc(CC(C)C)cc(Nc2ncc(Nc3ccncc3)cn2)c1] |
| A41 | 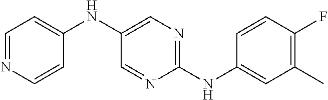 [SMILES: Cc1cc(Nc2ncc(Nc3ccncc3)cn2)ccc1F] |
| A42 | 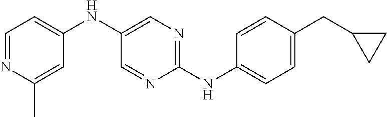 [SMILES: Cc1cc(Nc2cnc(Nc3ccc(CC4CC4)cc3)nc2)ccn1] |
| A43 | 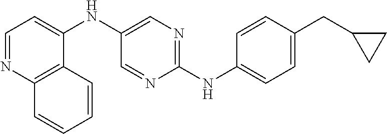 [SMILES: c1ccc2c(Nc3cnc(Nc4ccc(CC5CC5)cc4)nc3)ccnc2c1] |
| A44 | 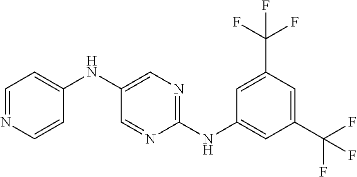 [SMILES: FC(F)(F)c1cc(Nc2ncc(Nc3ccncc3)cn2)cc(C(F)(F)F)c1] |
| A45 | 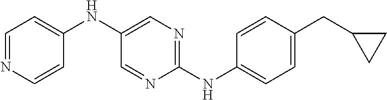 [SMILES: c1cc(Nc2cnc(Nc3ccc(CC4CC4)cc3)nc2)ccn1] |
| A46 | 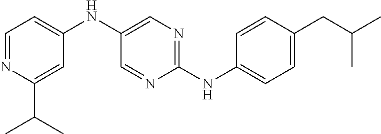 [SMILES: CC(C)Cc1ccc(Nc2ncc(Nc3ccnc(C(C)C)c3)cn2)cc1] |
| A47 | 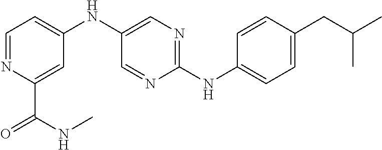 [SMILES: CNC(=O)c1cc(Nc2cnc(Nc3ccc(CC(C)C)cc3)nc2)ccn1] |
| A48 | 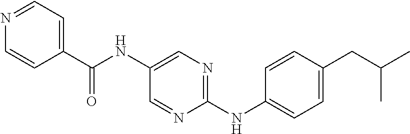 [SMILES: CC(C)Cc1ccc(Nc2ncc(NC(=O)c3ccncc3)cn2)cc1] |
| A49 | 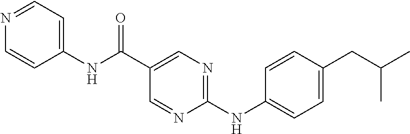 [SMILES: CC(C)Cc1ccc(Nc2ncc(C(=O)Nc3ccncc3)cn2)cc1] |
| A50 | 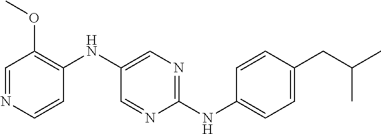 [SMILES: COc1cnccc1Nc1cnc(Nc2ccc(CC(C)C)cc2)nc1] |
| A51 | 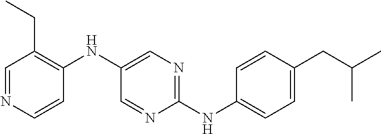 [SMILES: CCc1cnccc1Nc1cnc(Nc2ccc(CC(C)C)cc2)nc1] |
| A52 | 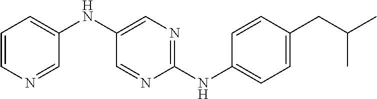 [SMILES: CC(C)Cc1ccc(Nc2ncc(Nc3cccnc3)cn2)cc1] |
| A53 | 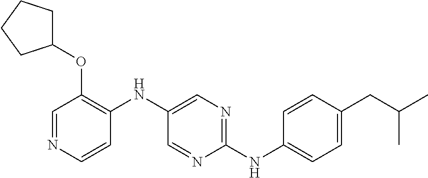 [SMILES: CC(C)Cc1ccc(Nc2ncc(Nc3ccncc3OC3CCCC3)cn2)cc1] |
| A54 | 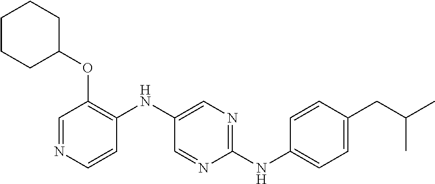 [SMILES: CC(C)Cc1ccc(Nc2ncc(Nc3ccncc3OC3CCCCC3)cn2)cc1] |
| A55 | 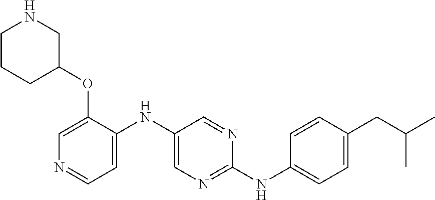 [SMILES: CC(C)Cc1ccc(Nc2ncc(Nc3ccncc3OC3CCCNC3)cn2)cc1] |
| A56 | 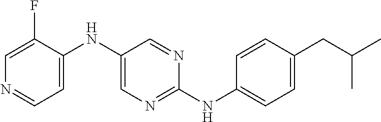 [SMILES: CC(C)Cc1ccc(Nc2ncc(Nc3ccncc3F)cn2)cc1] |
| A57 | 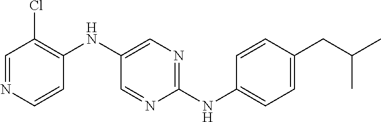 [SMILES: CC(C)Cc1ccc(Nc2ncc(Nc3ccncc3Cl)cn2)cc1] |
| A58 | 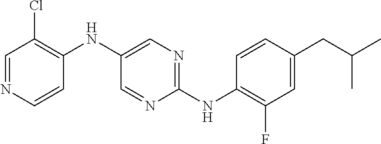 [SMILES: CC(C)Cc1ccc(Nc2ncc(Nc3ccncc3Cl)cn2)c(F)c1] |
| A59 | 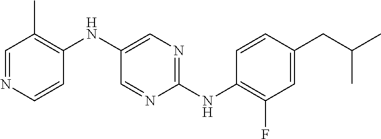 [SMILES: Cc1cnccc1Nc1cnc(Nc2ccc(CC(C)C)cc2F)nc1] |
| A60 | 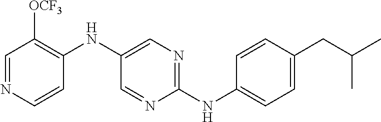 [SMILES: CC(C)Cc1ccc(Nc2ncc(Nc3ccncc3OC(F)(F)F)cn2)cc1] |
| A61 | 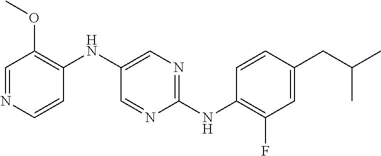 [SMILES: COc1cnccc1Nc1cnc(Nc2ccc(CC(C)C)cc2F)nc1] |
| A62 | 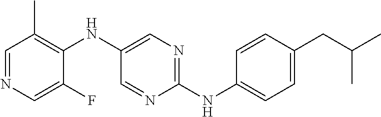 [SMILES: Cc1cncc(F)c1Nc1cnc(Nc2ccc(CC(C)C)cc2)nc1] |
| A63 | 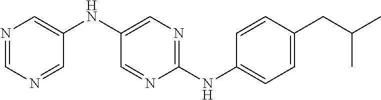 [SMILES: CC(C)Cc1ccc(Nc2ncc(Nc3cncnc3)cn2)cc1] |
| A64 | 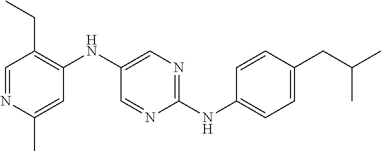 [SMILES: CCc1cnc(C)cc1Nc1cnc(Nc2ccc(CC(C)C)cc2)nc1] |
| A65 | 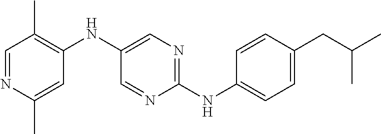 [SMILES: Cc1cc(Nc2cnc(Nc3ccc(CC(C)C)cc3)nc2)c(C)cn1] |
| A66 | 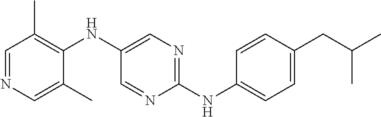 [SMILES: Cc1cncc(C)c1Nc1cnc(Nc2ccc(CC(C)C)cc2)nc1] |
| A67 | 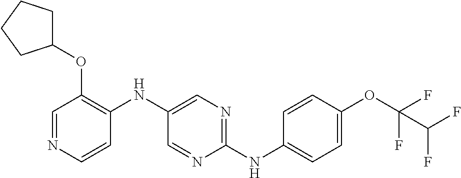 [SMILES: FC(F)C(F)(F)Oc1ccc(Nc2ncc(Nc3ccncc3OC3CCCC3)cn2)cc1] |
| A68 | 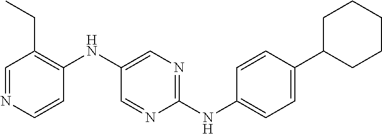 [SMILES: CCc1cnccc1Nc1cnc(Nc2ccc(C3CCCCC3)cc2)nc1] |
| A69 | 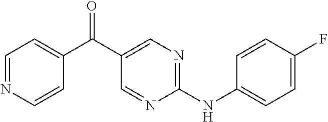 [SMILES: O=C(c1ccncc1)c1cnc(Nc2ccc(F)cc2)nc1] |
| A70 | 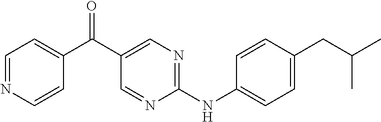 [SMILES: CC(C)Cc1ccc(Nc2ncc(C(=O)c3ccncc3)cn2)cc1] |
| A71 | 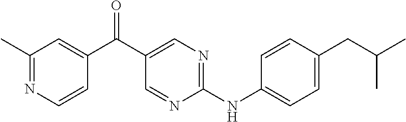 [SMILES: Cc1cc(C(=O)c2cnc(Nc3ccc(CC(C)C)cc3)nc2)ccn1] |
| A72 | 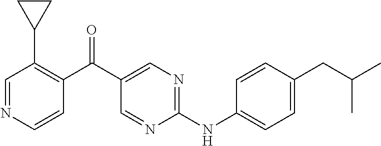 [SMILES: CC(C)Cc1ccc(Nc2ncc(C(=O)c3ccncc3C3CC3)cn2)cc1] |
| A73 | 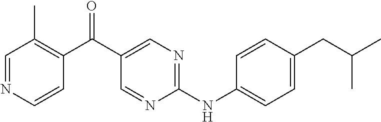 [SMILES: Cc1cnccc1C(=O)c1cnc(Nc2ccc(CC(C)C)cc2)nc1] |
| A74 | 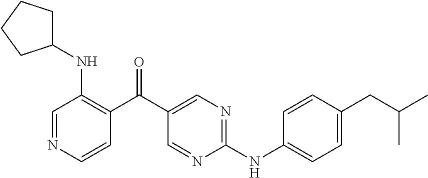 [SMILES: CC(C)Cc1ccc(Nc2ncc(C(=O)c3ccncc3NC3CCCC3)cn2)cc1] |
| A75 | 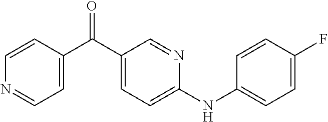 [SMILES: O=C(c1ccncc1)c1ccc(Nc2ccc(F)cc2)nc1] |
| A76 | 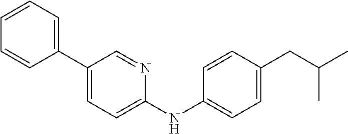 [SMILES: CC(C)Cc1ccc(Nc2ccc(-c3ccccc3)cn2)cc1] |
| A77 | 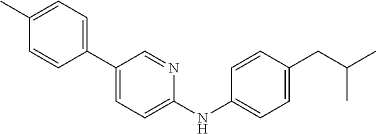 [SMILES: Cc1ccc(-c2ccc(Nc3ccc(CC(C)C)cc3)nc2)cc1] |
| A78 | 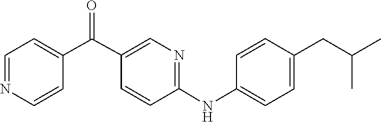 [SMILES: CC(C)Cc1ccc(Nc2ccc(C(=O)c3ccncc3)cn2)cc1] |
| A79 | 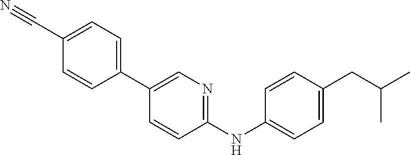 [SMILES: CC(C)Cc1ccc(Nc2ccc(-c3ccc(C#N)cc3)cn2)cc1] |
| A80 | 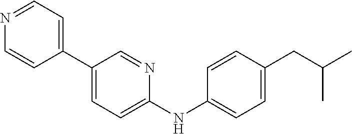 [SMILES: CC(C)Cc1ccc(Nc2ccc(-c3ccncc3)cn2)cc1] |
| A81 | 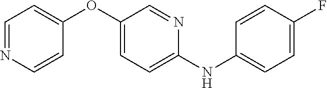 [SMILES: Fc1ccc(Nc2ccc(Oc3ccncc3)cn2)cc1] |
| A82 | 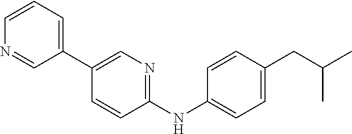 [SMILES: CC(C)Cc1ccc(Nc2ccc(-c3cccnc3)cn2)cc1] |
| A83 | 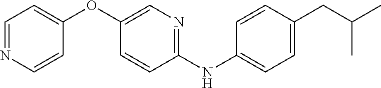 [SMILES: CC(C)Cc1ccc(Nc2ccc(Oc3ccncc3)cn2)cc1] |
| A84 | 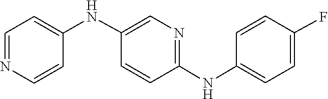 [SMILES: Fc1ccc(Nc2ccc(Nc3ccncc3)cn2)cc1] |
| A85 | 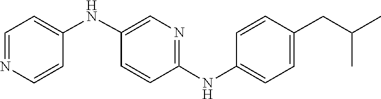 [SMILES: CC(C)Cc1ccc(Nc2ccc(Nc3ccncc3)cn2)cc1] |
| A86 | 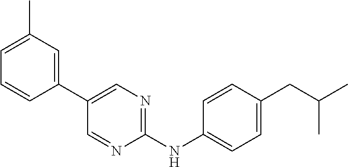 [SMILES: Cc1cccc(-c2cnc(Nc3ccc(CC(C)C)cc3)nc2)c1] |
| A87 | 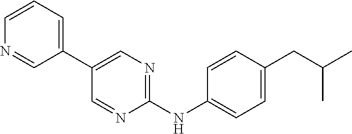 [SMILES: CC(C)Cc1ccc(Nc2ncc(-c3cccnc3)cn2)cc1] |
| A88 | 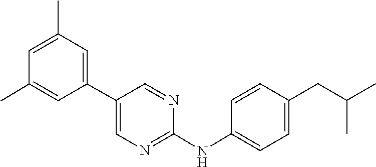 [SMILES: Cc1cc(C)cc(-c2cnc(Nc3ccc(CC(C)C)cc3)nc2)c1] |
| A89 | 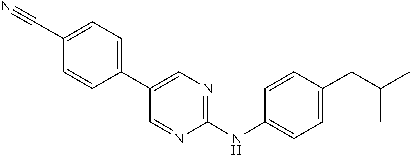 [SMILES: CC(C)Cc1ccc(Nc2ncc(-c3ccc(C#N)cc3)cn2)cc1] |
| A90 | 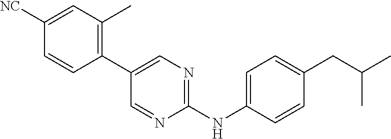 [SMILES: Cc1cc(C#N)ccc1-c1cnc(Nc2ccc(CC(C)C)cc2)nc1] |
| A91 | 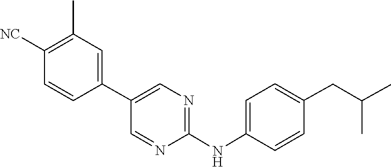 [SMILES: Cc1cc(-c2cnc(Nc3ccc(CC(C)C)cc3)nc2)ccc1C#N] |
| A92 | 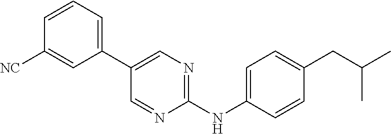 [SMILES: CC(C)Cc1ccc(Nc2ncc(-c3cccc(C#N)c3)cn2)cc1] |
| A93 | 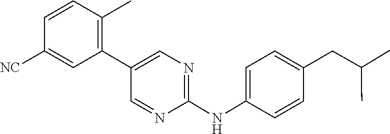 [SMILES: Cc1ccc(C#N)cc1-c1cnc(Nc2ccc(CC(C)C)cc2)nc1] |
| A94 | 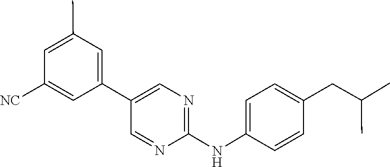 [SMILES: Cc1cc(C#N)cc(-c2cnc(Nc3ccc(CC(C)C)cc3)nc2)c1] |
| A95 | 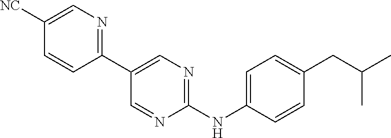 [SMILES: CC(C)Cc1ccc(Nc2ncc(-c3ccc(C#N)cn3)cn2)cc1] |
| A96 | 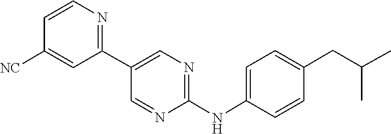 [SMILES: CC(C)Cc1ccc(Nc2ncc(-c3cc(C#N)ccn3)cn2)cc1] |
| A97 | 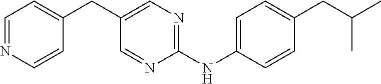 [SMILES: CC(C)Cc1ccc(Nc2ncc(Cc3ccncc3)cn2)cc1] |
| A98 | 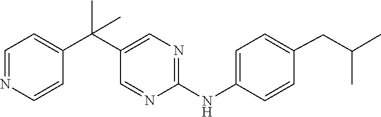 [SMILES: CC(C)Cc1ccc(Nc2ncc(C(C)(C)c3ccncc3)cn2)cc1] |
| A99 | 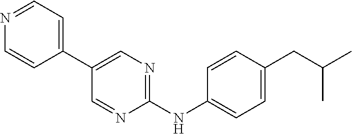 [SMILES: CC(C)Cc1ccc(Nc2ncc(-c3ccncc3)cn2)cc1] |
| A100 | 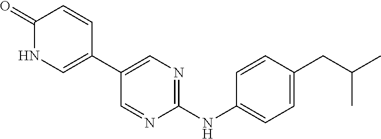 [SMILES: CC(C)Cc1ccc(Nc2ncc(-c3ccc(=O)[nH]c3)cn2)cc1] |
| A101 | 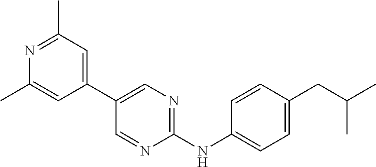 [SMILES: Cc1cc(-c2cnc(Nc3ccc(CC(C)C)cc3)nc2)cc(C)n1] |
| A102 | 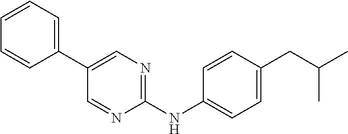 [SMILES: CC(C)Cc1ccc(Nc2ncc(-c3ccccc3)cn2)cc1] |
| A103 | 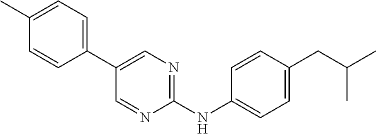 [SMILES: Cc1ccc(-c2cnc(Nc3ccc(CC(C)C)cc3)nc2)cc1] |
| A104 | 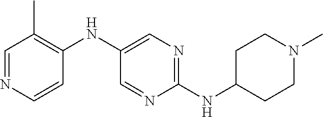 [SMILES: Cc1cnccc1Nc1cnc(NC2CCN(C)CC2)nc1] |
| A105 | 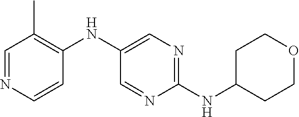 [SMILES: Cc1cnccc1Nc1cnc(NC2CCOCC2)nc1] |
| A106 | 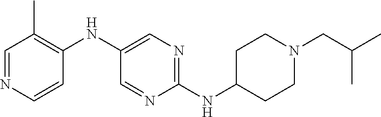 [SMILES: Cc1cnccc1Nc1cnc(NC2CCN(CC(C)C)CC2)nc1] |
| A107 | 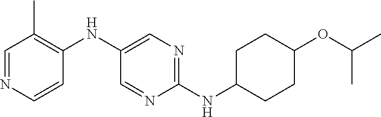 [SMILES: Cc1cnccc1Nc1cnc(NC2CCC(OC(C)C)CC2)nc1] |
| A108 | 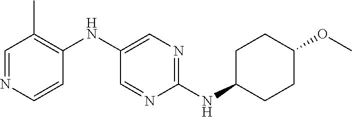 [SMILES: CO[C@H]1CC[C@H](Nc2ncc(Nc3ccncc3C)cn2)CC1] |
| A109 | 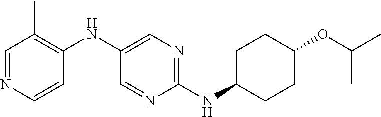 [SMILES: Cc1cnccc1Nc1cnc(N[C@H]2CC[C@H](OC(C)C)CC2)nc1] |
| A110 | 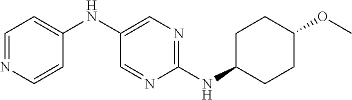 [SMILES: CO[C@H]1CC[C@H](Nc2ncc(Nc3ccncc3)cn2)CC1] |
| A111 | 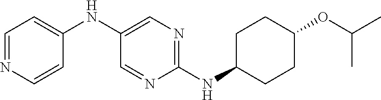 [SMILES: CC(C)O[C@H]1CC[C@H](Nc2ncc(Nc3ccncc3)cn2)CC1] |
| A112 | 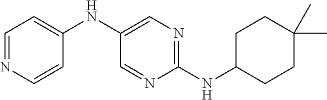 [SMILES: CC1(C)CCC(Nc2ncc(Nc3ccncc3)cn2)CC1] |
| A113 | 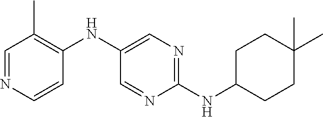 [SMILES: Cc1cnccc1Nc1cnc(NC2CCC(C)(C)CC2)nc1] |
| A114 | 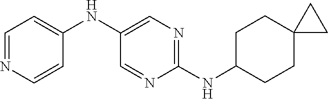 [SMILES: c1cc(Nc2cnc(NC3CCC4(CC3)CC4)nc2)ccn1] |
| A115 | 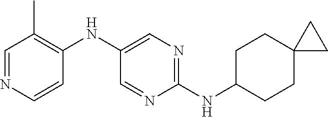 [SMILES: Cc1cnccc1Nc1cnc(NC2CCC3(CC2)CC3)nc1] |
| A116 | 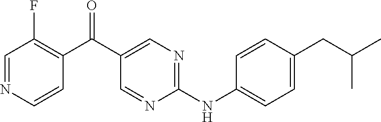 [SMILES: CC(C)Cc1ccc(Nc2ncc(C(=O)c3ccncc3F)cn2)cc1] |
| A117 | 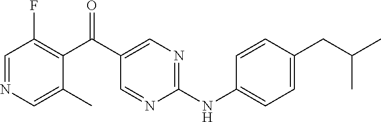 [SMILES: Cc1cncc(F)c1C(=O)c1cnc(Nc2ccc(CC(C)C)cc2)nc1] |
| A118 | 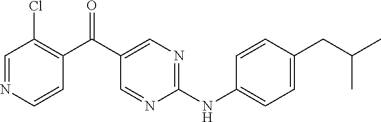 [SMILES: CC(C)Cc1ccc(Nc2ncc(C(=O)c3ccncc3Cl)cn2)cc1] |
| A119 | 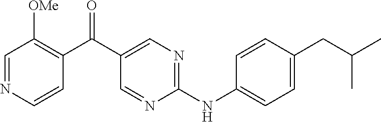 [SMILES: COc1cnccc1C(=O)c1cnc(Nc2ccc(CC(C)C)cc2)nc1] |
| A120 | 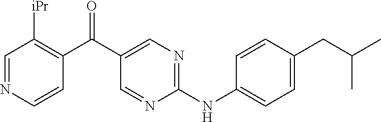 [SMILES: CC(C)Cc1ccc(Nc2ncc(C(=O)c3ccncc3C(C)C)cn2)cc1] |
| A121 | 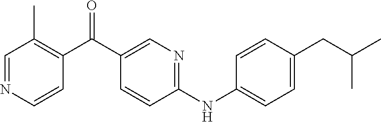 [SMILES: Cc1cnccc1C(=O)c1ccc(Nc2ccc(CC(C)C)cc2)nc1] |
| A122 | 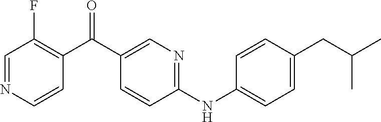 [SMILES: CC(C)Cc1ccc(Nc2ccc(C(=O)c3ccncc3F)cn2)cc1] |
| A123 | 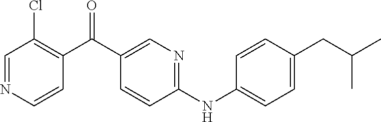 [SMILES: CC(C)Cc1ccc(Nc2ccc(C(=O)c3ccncc3Cl)cn2)cc1] |
| A124 | 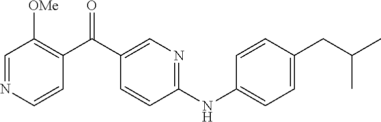 [SMILES: COc1cnccc1C(=O)c1ccc(Nc2ccc(CC(C)C)cc2)nc1] |
| A125 | 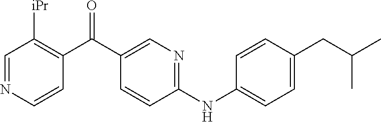 [SMILES: CC(C)Cc1ccc(Nc2ccc(C(=O)c3ccncc3C(C)C)cn2)cc1] |
| A126 | 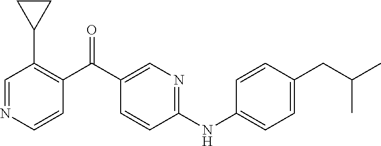 [SMILES: CC(C)Cc1ccc(Nc2ccc(C(=O)c3ccncc3C3CC3)cn2)cc1] |
| A128 | 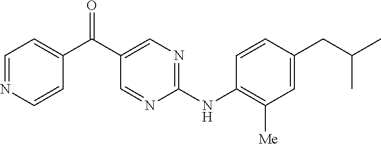 [SMILES: Cc1cc(CC(C)C)ccc1Nc1ncc(C(=O)c2ccncc2)cn1] |
| A129 | 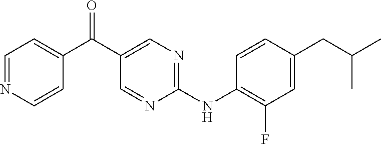 [SMILES: CC(C)Cc1ccc(Nc2ncc(C(=O)c3ccncc3)cn2)c(F)c1] |
| A130 | 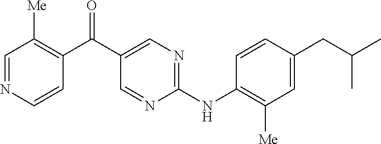 [SMILES: Cc1cc(CC(C)C)ccc1Nc1ncc(C(=O)c2ccncc2C)cn1] |
| A131 | 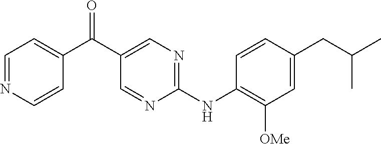 [SMILES: COc1cc(CC(C)C)ccc1Nc1ncc(C(=O)c2ccncc2)cn1] |
| A132 | 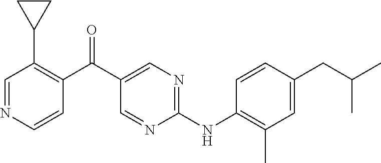 [SMILES: Cc1cc(CC(C)C)ccc1Nc1ncc(C(=O)c2ccncc2C2CC2)cn1] |
| A133 | 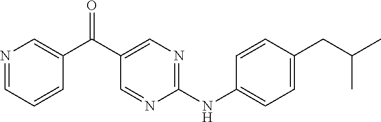 [SMILES: CC(C)Cc1ccc(Nc2ncc(C(=O)c3cccnc3)cn2)cc1] |
| A134 | 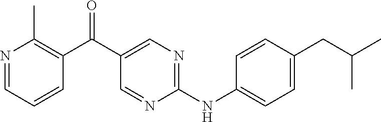 [SMILES: Cc1ncccc1C(=O)c1cnc(Nc2ccc(CC(C)C)cc2)nc1] |
| A135 | 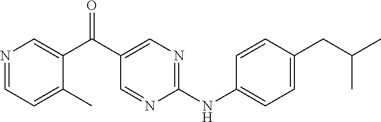 [SMILES: Cc1ccncc1C(=O)c1cnc(Nc2ccc(CC(C)C)cc2)nc1] |
| A136 | 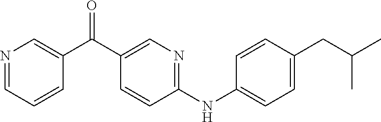 [SMILES: CC(C)Cc1ccc(Nc2ccc(C(=O)c3cccnc3)cn2)cc1] |
| A137 | 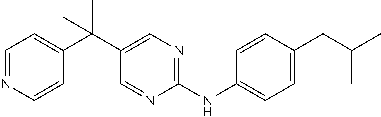 [SMILES: CC(C)Cc1ccc(Nc2ncc(C(C)(C)c3ccncc3)cn2)cc1] |
| A138 | 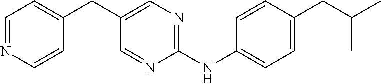 [SMILES: CC(C)Cc1ccc(Nc2ncc(Cc3ccncc3)cn2)cc1] |
| A139 | 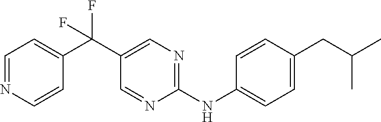 [SMILES: CC(C)Cc1ccc(Nc2ncc(C(F)(F)c3ccncc3)cn2)cc1] |
| A140 | 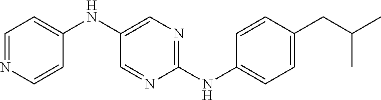 [SMILES: CC(C)Cc1ccc(Nc2ncc(Nc3ccncc3)cn2)cc1] |
| A141 | 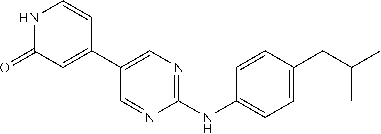 [SMILES: CC(C)Cc1ccc(Nc2ncc(-c3cc[nH]c(=O)c3)cn2)cc1] |
| A142 | 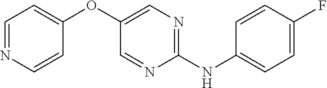 [SMILES: Fc1ccc(Nc2ncc(Oc3ccncc3)cn2)cc1] |
| A143 | 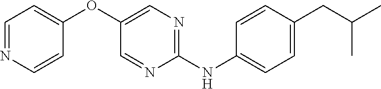 [SMILES: CC(C)Cc1ccc(Nc2ncc(Oc3ccncc3)cn2)cc1] |
| A144 | 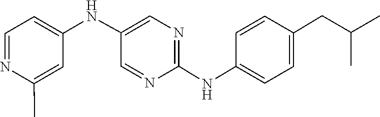 [SMILES: Cc1cc(Nc2cnc(Nc3ccc(CC(C)C)cc3)nc2)ccn1] |
| A145 | 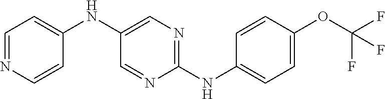 [SMILES: FC(F)(F)Oc1ccc(Nc2ncc(Nc3ccncc3)cn2)cc1] |
| A146 | 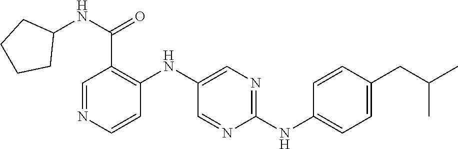 [SMILES: CC(C)Cc1ccc(Nc2ncc(Nc3ccncc3C(=O)NC3CCCC3)cn2)cc1] |
| A147 | 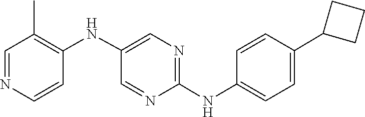 [SMILES: Cc1cnccc1Nc1cnc(Nc2ccc(C3CCC3)cc2)nc1] |
| A148 | 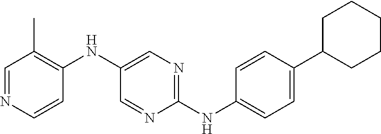 [SMILES: Cc1cnccc1Nc1cnc(Nc2ccc(C3CCCCC3)cc2)nc1] |
| A149 | 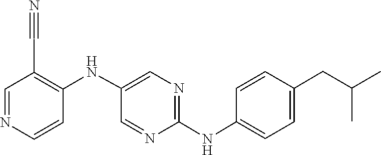 [SMILES: CC(C)Cc1ccc(Nc2ncc(Nc3ccncc3C#N)cn2)cc1] |
| A150 | 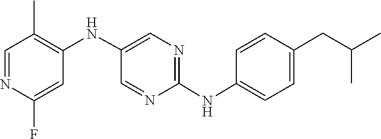 [SMILES: Cc1cnc(F)cc1Nc1cnc(Nc2ccc(CC(C)C)cc2)nc1] |
| A151 | 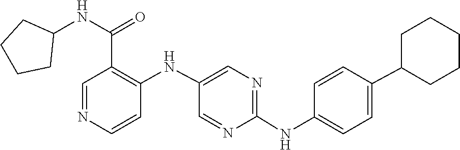 [SMILES: O=C(NC1CCCC1)c1cnccc1Nc1cnc(Nc2ccc(C3CCCCC3)cc2)nc1] |
| A152 | 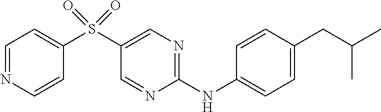 [SMILES: CC(C)Cc1ccc(Nc2ncc(S(=O)(=O)c3ccncc3)cn2)cc1] |
| A153 | 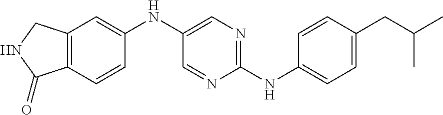 [SMILES: CC(C)Cc1ccc(Nc2ncc(Nc3ccc4c(c3)CNC4=O)cn2)cc1] |
| A154 | 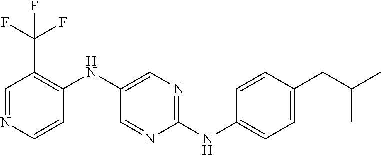 [SMILES: CC(C)Cc1ccc(Nc2ncc(Nc3ccncc3C(F)(F)F)cn2)cc1] |
| A155 | 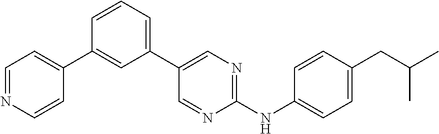 [SMILES: CC(C)Cc1ccc(Nc2ncc(-c3cccc(-c4ccncc4)c3)cn2)cc1] |
| A156 | 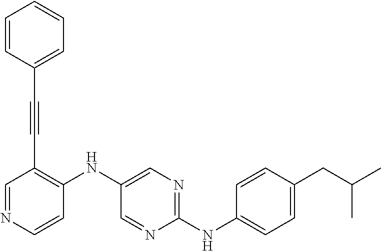 [SMILES: CC(C)Cc1ccc(Nc2ncc(Nc3ccncc3C#Cc3ccccc3)cn2)cc1] |
| A157 | 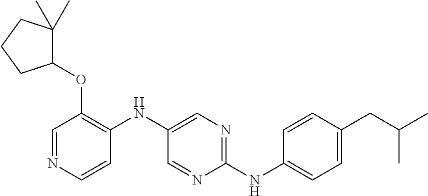 [SMILES: CC(C)Cc1ccc(Nc2ncc(Nc3ccncc3OC3CCCC3(C)C)cn2)cc1] |
| A158 | 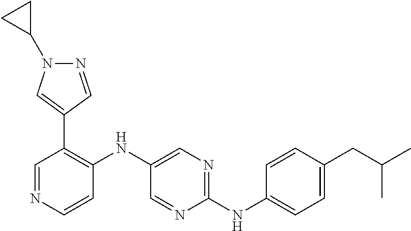 [SMILES: CC(C)Cc1ccc(Nc2ncc(Nc3ccncc3-c3cnn(C4CC4)c3)cn2)cc1] |
| A159 | 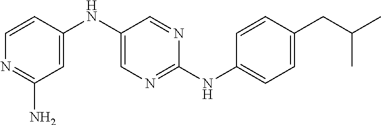 [SMILES: CC(C)Cc1ccc(Nc2ncc(Nc3ccnc(N)c3)cn2)cc1] |
| A160 | 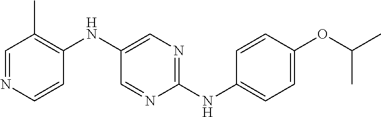 [SMILES: Cc1cnccc1Nc1cnc(Nc2ccc(OC(C)C)cc2)nc1] |
| A161 | 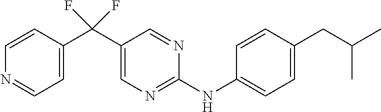 [SMILES: CC(C)Cc1ccc(Nc2ncc(C(F)(F)c3ccncc3)cn2)cc1] |
| A162 | 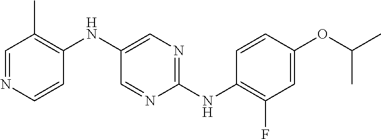 [SMILES: Cc1cnccc1Nc1cnc(Nc2ccc(OC(C)C)cc2F)nc1] |
| A163 | 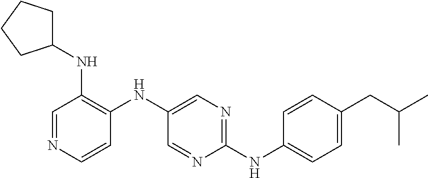 [SMILES: CC(C)Cc1ccc(Nc2ncc(Nc3ccncc3NC3CCCC3)cn2)cc1] |
| A164 | 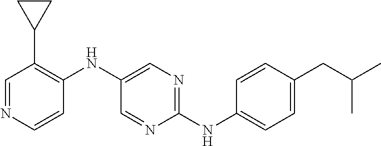 [SMILES: CC(C)Cc1ccc(Nc2ncc(Nc3ccncc3C3CC3)cn2)cc1] |
| A165 | 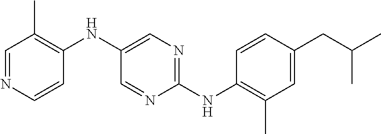 [SMILES: Cc1cnccc1Nc1cnc(Nc2ccc(CC(C)C)cc2C)nc1] |
| A166 | 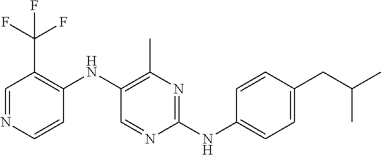 [SMILES: Cc1nc(Nc2ccc(CC(C)C)cc2)ncc1Nc1ccncc1C(F)(F)F] |
| A167 | 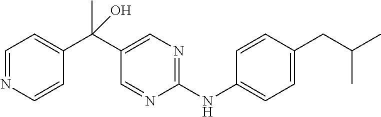 [SMILES: CC(C)Cc1ccc(Nc2ncc(C(C)(O)c3ccncc3)cn2)cc1] |
| A168 | 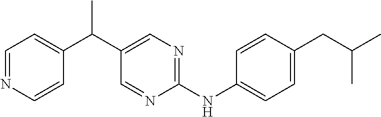 [SMILES: CC(C)Cc1ccc(Nc2ncc(C(C)c3ccncc3)cn2)cc1] |
| A169 | 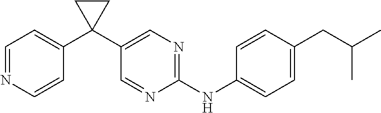 [SMILES: CC(C)Cc1ccc(Nc2ncc(C3(c4ccncc4)CC3)cn2)cc1] |
| A170 | 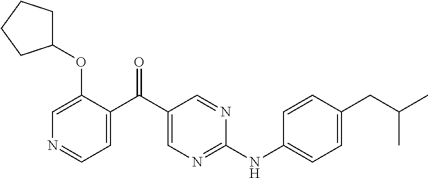 [SMILES: CC(C)Cc1ccc(Nc2ncc(C(=O)c3ccncc3OC3CCCC3)cn2)cc1] |
| A171 | 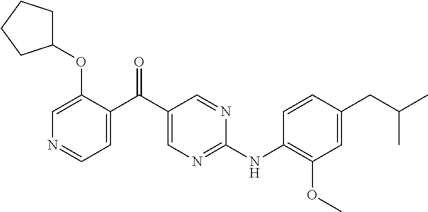 [SMILES: COc1cc(CC(C)C)ccc1Nc1ncc(C(=O)c2ccncc2OC2CCCC2)cn1] |
| A172 | 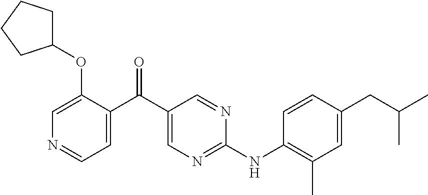 [SMILES: Cc1cc(CC(C)C)ccc1Nc1ncc(C(=O)c2ccncc2OC2CCCC2)cn1] |
| A173 | 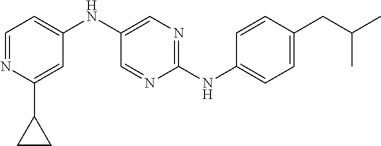 [SMILES: CC(C)Cc1ccc(Nc2ncc(Nc3ccnc(C4CC4)c3)cn2)cc1] |
| A174 | 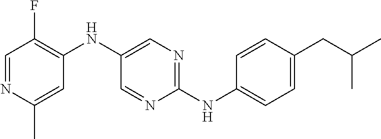 [SMILES: Cc1cc(Nc2cnc(Nc3ccc(CC(C)C)cc3)nc2)c(F)cn1] |
| A175 | 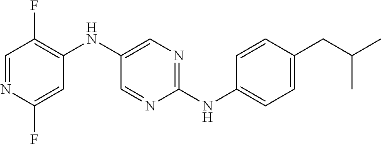 [SMILES: CC(C)Cc1ccc(Nc2ncc(Nc3cc(F)ncc3F)cn2)cc1] |
| A176 | 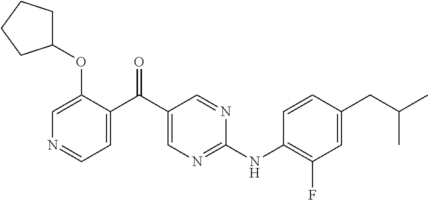 [SMILES: CC(C)Cc1ccc(Nc2ncc(C(=O)c3ccncc3OC3CCCC3)cn2)c(F)c1] |
| A177 | 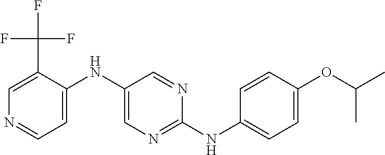 [SMILES: CC(C)Oc1ccc(Nc2ncc(Nc3ccncc3C(F)(F)F)cn2)cc1] |
| A178 | 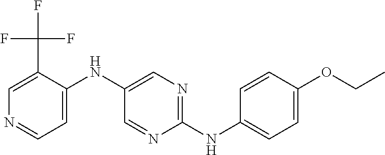 [SMILES: CCOc1ccc(Nc2ncc(Nc3ccncc3C(F)(F)F)cn2)cc1] |
| A179 | 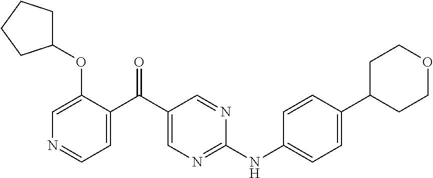 [SMILES: O=C(c1cnc(Nc2ccc(C3CCOCC3)cc2)nc1)c1ccncc1OC1CCCC1] |
| A180 | 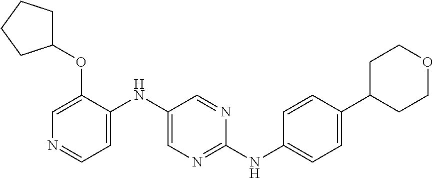 [SMILES: c1cc(Nc2cnc(Nc3ccc(C4CCOCC4)cc3)nc2)c(OC2CCCC2)cn1] |
| A181 | 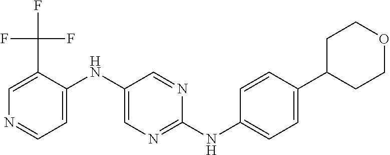 [SMILES: FC(F)(F)c1cnccc1Nc1cnc(Nc2ccc(C3CCOCC3)cc2)nc1] |
| A182 | 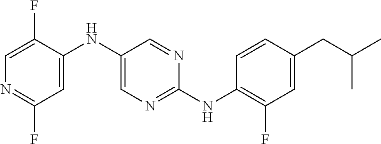 [SMILES: CC(C)Cc1ccc(Nc2ncc(Nc3cc(F)ncc3F)cn2)c(F)c1] |
| A183 | 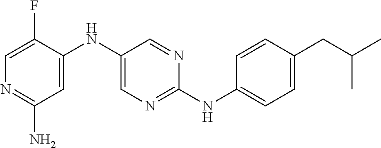 [SMILES: CC(C)Cc1ccc(Nc2ncc(Nc3cc(N)ncc3F)cn2)cc1] |
| A184 | 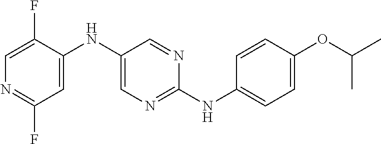 [SMILES: CC(C)Oc1ccc(Nc2ncc(Nc3cc(F)ncc3F)cn2)cc1] |
| A185 | 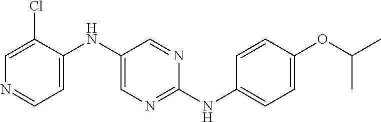 [SMILES: CC(C)Oc1ccc(Nc2ncc(Nc3ccncc3Cl)cn2)cc1] |
| A186 | 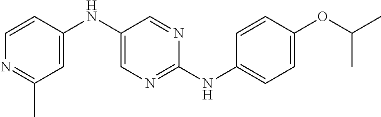 [SMILES: Cc1cc(Nc2cnc(Nc3ccc(OC(C)C)cc3)nc2)ccn1] |
| A187 | 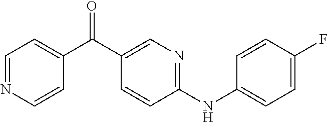 [SMILES: O=C(c1ccncc1)c1ccc(Nc2ccc(F)cc2)nc1] |
| A188 | 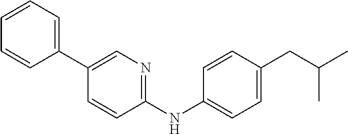 [SMILES: CC(C)Cc1ccc(Nc2ccc(-c3ccccc3)cn2)cc1] |
| A189 | 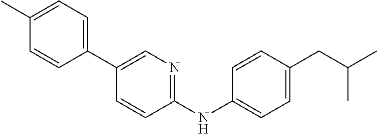 [SMILES: Cc1ccc(-c2ccc(Nc3ccc(CC(C)C)cc3)nc2)cc1] |
| A190 | 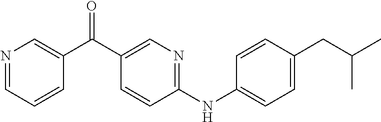 [SMILES: CC(C)Cc1ccc(Nc2ccc(C(=O)c3cccnc3)cn2)cc1] |
| A191 | 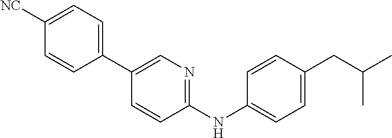 [SMILES: CC(C)Cc1ccc(Nc2ccc(-c3ccc(C#N)cc3)cn2)cc1] |
| A192 | 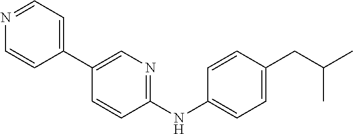 [SMILES: CC(C)Cc1ccc(Nc2ccc(-c3ccncc3)cn2)cc1] |
| A193 | 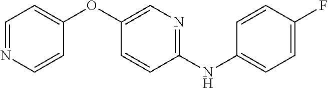 [SMILES: Fc1ccc(Nc2ccc(Oc3ccncc3)cn2)cc1] |
| A194 | 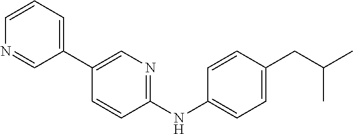 [SMILES: CC(C)Cc1ccc(Nc2ccc(-c3cccnc3)cn2)cc1] |
| A195 | 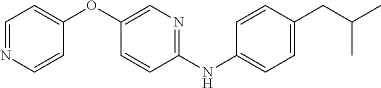 [SMILES: CC(C)Cc1ccc(Nc2ccc(Oc3ccncc3)cn2)cc1] |
| A196 | 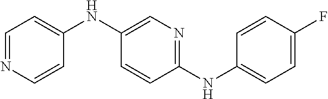 [SMILES: Fc1ccc(Nc2ccc(Nc3ccncc3)cn2)cc1] |
| A197 | 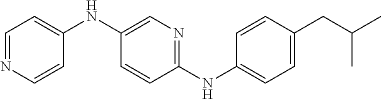 [SMILES: CC(C)Cc1ccc(Nc2ccc(Nc3ccncc3)cn2)cc1] |
| A198 | 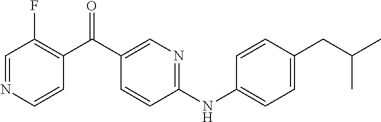 [SMILES: CC(C)Cc1ccc(Nc2ccc(C(=O)c3ccncc3F)cn2)cc1] |
| A199 | 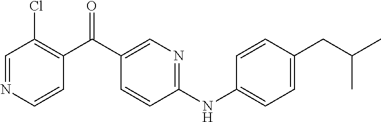 [SMILES: CC(C)Cc1ccc(Nc2ccc(C(=O)c3ccncc3Cl)cn2)cc1] |
| A200 | 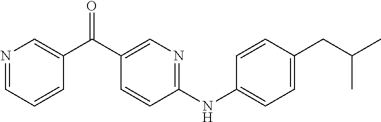 [SMILES: CC(C)Cc1ccc(Nc2ccc(C(=O)c3cccnc3)cn2)cc1] |
| A201 | 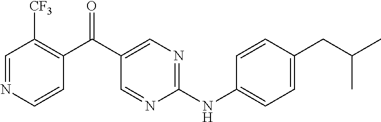 [SMILES: CC(C)Cc1ccc(Nc2ncc(C(=O)c3ccncc3C(F)(F)F)cn2)cc1] |
| A202 | 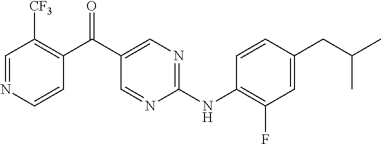 [SMILES: CC(C)Cc1ccc(Nc2ncc(C(=O)c3ccncc3C(F)(F)F)cn2)c(F)c1] |
| A203 | 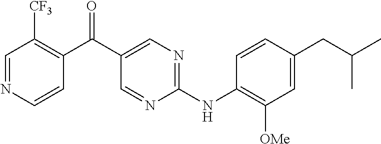 [SMILES: COc1cc(CC(C)C)ccc1Nc1ncc(C(=O)c2ccncc2C(F)(F)F)cn1] |
| A204 | 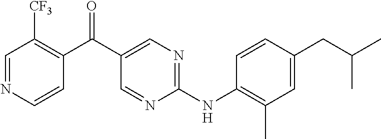 [SMILES: Cc1cc(CC(C)C)ccc1Nc1ncc(C(=O)c2ccncc2C(F)(F)F)cn1] |
| A205 | 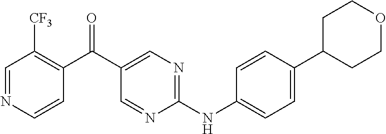 [SMILES: O=C(c1cnc(Nc2ccc(C3CCOCC3)cc2)nc1)c1ccncc1C(F)(F)F] |
| A206 | 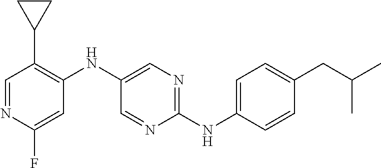 [SMILES: CC(C)Cc1ccc(Nc2ncc(Nc3cc(F)ncc3C3CC3)cn2)cc1] |
| A207 | 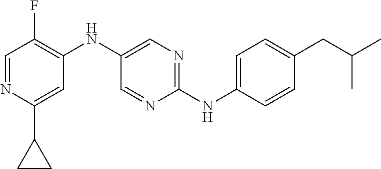 [SMILES: CC(C)Cc1ccc(Nc2ncc(Nc3cc(C4CC4)ncc3F)cn2)cc1] |
| A208 | 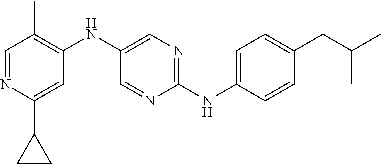 [SMILES: Cc1cnc(C2CC2)cc1Nc1cnc(Nc2ccc(CC(C)C)cc2)nc1] |
| A209 | 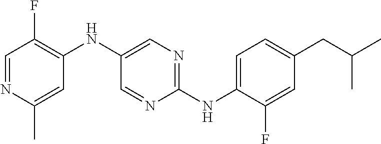 [SMILES: Cc1cc(Nc2cnc(Nc3ccc(CC(C)C)cc3F)nc2)c(F)cn1] |
| A210 | 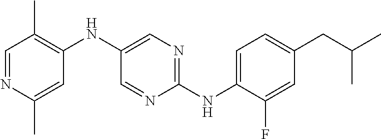 [SMILES: Cc1cc(Nc2cnc(Nc3ccc(CC(C)C)cc3F)nc2)c(C)cn1] |
| A211 | 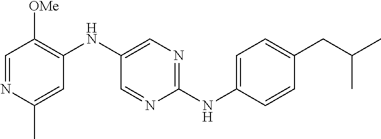 [SMILES: COc1cnc(C)cc1Nc1cnc(Nc2ccc(CC(C)C)cc2)nc1] |
| A212 | 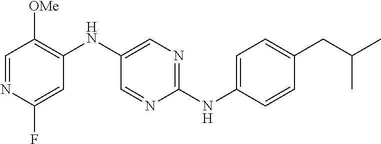 [SMILES: COc1cnc(F)cc1Nc1cnc(Nc2ccc(CC(C)C)cc2)nc1] |
| A213 | 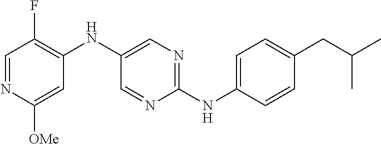 [SMILES: COc1cc(Nc2cnc(Nc3ccc(CC(C)C)cc3)nc2)c(F)cn1] |
| A214 | 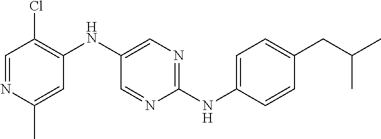 [SMILES: Cc1cc(Nc2cnc(Nc3ccc(CC(C)C)cc3)nc2)c(Cl)cn1] |
| A215 | 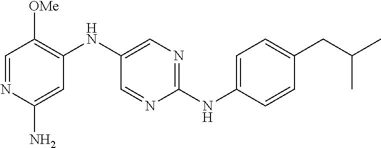 [SMILES: COc1cnc(N)cc1Nc1cnc(Nc2ccc(CC(C)C)cc2)nc1] |
| A216 | 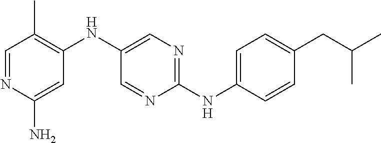 [SMILES: Cc1cnc(N)cc1Nc1cnc(Nc2ccc(CC(C)C)cc2)nc1] |
| A217 | 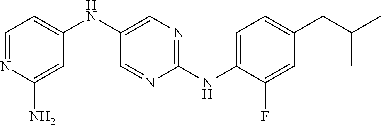 [SMILES: CC(C)Cc1ccc(Nc2ncc(Nc3ccnc(N)c3)cn2)c(F)c1] |
| A218 | 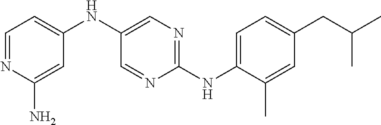 [SMILES: Cc1cc(CC(C)C)ccc1Nc1ncc(Nc2ccnc(N)c2)cn1] |
| A219 | 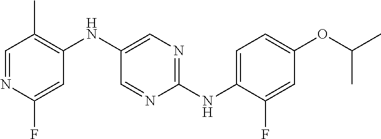 [SMILES: Cc1cnc(F)cc1Nc1cnc(Nc2ccc(OC(C)C)cc2F)nc1] |
| A220 | 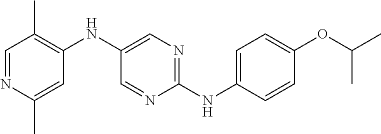 [SMILES: Cc1cc(Nc2cnc(Nc3ccc(OC(C)C)cc3)nc2)c(C)cn1] |
| A221 | 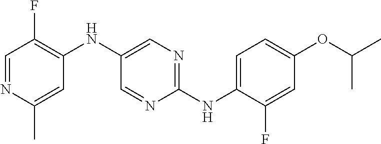 [SMILES: Cc1cc(Nc2cnc(Nc3ccc(OC(C)C)cc3F)nc2)c(F)cn1] |
| A222 | 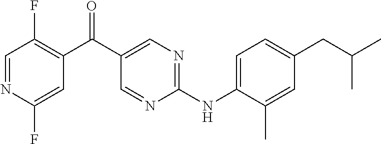 [SMILES: Cc1cc(CC(C)C)ccc1Nc1ncc(C(=O)c2cc(F)ncc2F)cn1] |
| A223 | 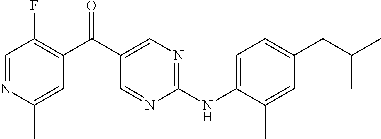 [SMILES: Cc1cc(C(=O)c2cnc(Nc3ccc(CC(C)C)cc3C)nc2)c(F)cn1] |
| A224 | 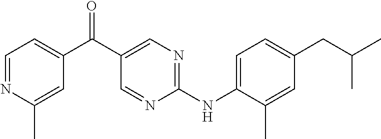 [SMILES: Cc1cc(C(=O)c2cnc(Nc3ccc(CC(C)C)cc3C)nc2)ccn1] |
| A225 | 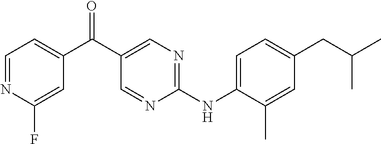 [SMILES: Cc1cc(CC(C)C)ccc1Nc1ncc(C(=O)c2ccnc(F)c2)cn1] |
| A226 | 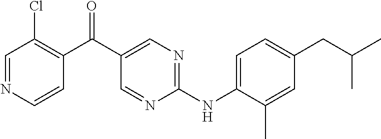 [SMILES: Cc1cc(CC(C)C)ccc1Nc1ncc(C(=O)c2ccncc2Cl)cn1] |

The compounds described herein can exist in a tautomer form. For example, when Z¹ is N, Z² is CH, and R¹ is ortho to the N and is a hydroxyl group, the form can alternatively be depicted as either of the two structures below:

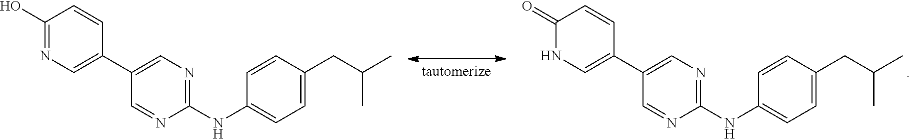 [SMILES: CC(C)Cc1ccc(Nc2ncc(-c3ccc(O)nc3)cn2)cc1 ; CC(C)Cc1ccc(Nc2ncc(-c3ccc(=O)[nH]c3)cn2)cc1]

The compounds described herein can exist in free form, or, where appropriate, as salts. Those salts that are pharmaceutically acceptable are of particular interest since they are useful in administering the compounds described below for medical purposes. Salts that are not pharmaceutically acceptable are useful in manufacturing processes, for isolation and purification purposes, and in some instances, for use in separating stereoisomeric forms of the compounds described herein or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺ (C₁₋₄alkyl) 4 salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this disclosure includes mixtures/combinations of different pharmaceutically acceptable salts and also mixtures/combinations of compounds in free form and pharmaceutically acceptable salts.

### Preparation of Compounds Disclosed Herein

Synthesis of the compounds described herein can be done using any suitable method. The present disclosure also provides methods of preparing a compound described herein. The compounds described herein, and pharmaceutical salts thereof, all include a core structure including ring A linked to a pyridine/pyrimidine moiety, which is linked to an aryl group via linking moiety Y.

Illustrative procedures for preparing the disclosed compounds are described herein at the Examples.

### Pharmaceutical Compositions

The compounds described herein can be formulated into pharmaceutical compositions that further comprise a pharmaceutically acceptable carrier, diluent, adjuvant or vehicle. In some embodiments, the present disclosure relates to a pharmaceutical composition comprising a compound described herein, and a pharmaceutically acceptable carrier, diluent, adjuvant or vehicle. In some embodiments, the present disclosure includes a pharmaceutical composition comprising a safe and effective amount of a compound described herein or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating an influenza virus infection in a patient. The term “prophylactically effective amount” refers to an amount effective in preventing and/or substantially lessening the chances or the size of influenza virus infection outbreak.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any solvents, diluents, or other liquid vehicle, dispersion or suspension aids, surface active agents, isotonic agents, thickening or emulsifying agents, preservatives, solid binders, lubricants and the like, as suited to the particular dosage form desired. Remington s Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this disclosure. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer s solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Methods of Use

The compounds described herein or pharmaceutically acceptable salts thereof can be used to inhibit CDK19 activity, and thus can be used to treat conditions mediated by CDK19, such as cancer, more particularly, cancers having aberrant CDK19 activity and the compound or salt thereof can modify that aberrant activity (e.g., downregulation or inhibition of CDK19 activity). Suitable cancers which can be treated by the disclosed methods, include but are not limited to breast cancer, prostate cancer, cancer of the gastrointestinal tract (e.g., colorectal cancer), bladder cancer, sarcoma, cervical cancer, esophageal adenocarcinoma, acute myeloid leukemia, melanoma, glioma, and ovarian cancer. In some cases, the cancer is triple negative breast cancer.

As used herein, the terms “treat”, “treatment,” and “treating” refer to the reduction or amelioration of the progression, severity and/or duration of the CDK19 mediated condition (e.g., TNBC), or the amelioration of one or more symptoms (specifically, one or more discernible symptoms) of CDK19-mediated condition, resulting from the administration of one or more therapies (e.g., one or more therapeutic agents such as a compound or composition described herein).

As used herein, the term “inhibitor” as used in the context of CDK19, refers to a compound, or pharmaceutical composition that reduces the expression or activity of CDK19. Desirably, the compound or pharmaceutical composition selectively inhibits CDK19 expression or activity over that of CDK8.

As used here, the terms “decrease,” “reduced,” “reduction,” and “decreasing” are all used herein to refer to a decrease by at least 10% as compared to a reference level, for example a decrease by at least about 5%, at least about 10%, at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The disclosed compounds, or pharmaceutically acceptable salts thereof, are selective for CDK19 over other kinases (e.g., CDK8). For example, compounds of formula (I) when screened against a panel of other kinases (e.g., using KINOMEscan® assay), exhibit selectivity for inhibiting CDK19.

The inhibition of CDK19 activity can be measure by any suitable method known in the art. For example, any suitable enzyme inhibition assay (e.g., competitive binding assay) or functional cell-based assay can be used to measure CDK19 activity. An illustrative assay for measuring CDK19 activity is a FRET-based (Forster resonant energy transfer) assay.

In some embodiments, the disclosure provides a method of inhibiting cyclin dependent kinase 19 (CDK19) comprising contacting CDK19 with one or more of the disclosed compounds in an amount effective to inhibit CDK19.

In some embodiments, the disclosed compounds selectively inhibit CDK19 over CDK8. CDK8 inhibitory activity can be measured using any suitable inhibition assay, including FRET-based assays. In some embodiments, the compounds are at least 2 times more selective for CDK19 over CDK8. For example, in some embodiments the compounds are at least 3 times more selective for CDK19 (e.g., at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 times or more selective for CDK19 over CDK8). In some embodiments, the compounds are at least 20 times more selective for CDK19 over CDK8. In some embodiments, the compounds are 3.6 times more selective for CDK19 over CDK8, based upon IC₅₀ measurements. In some embodiments, the compounds are 22.4 times more selective for CDK19 over CDK8, based upon IC 50 measurements.

In some embodiments, the disclosed compounds have an IC₅₀ for CDK19 of less than 400 nM (e.g., 375, 350, 325, 300, 275, 250, 225 nm). In some embodiments, the compound has an IC₅₀ for CDK19 of less than 200 nM (e.g., 175, 150, 125, 100, 90, 80, 70, 60, 50, 40, 30, 20, 10, 5, 4, 3, 2, or less than 1 nM). In some embodiments, the compounds have an IC₅₀ for CDK19 of 20 nm. In some embodiments, the compounds haves an IC₅₀ for CDK19 of 2.5 nM.

In some embodiments, the disclosed compounds have a lethal dose 50 (LD₅₀) in a TNBC cell line of 500 nM or less (e.g., 500, 450, 400, 350, 300, 250, 200, 150, 100 nM or less). In some embodiments the disclosed compounds have a LD₅₀ in MDA-MB-231 TNBC cells of 180 nM, 178 nM, 158 nM, or 91 nM. In some embodiments, the disclosed compounds have a LD₅₀ in normal cells (e.g., human foreskin fibroblast cells) of 1,000 nM or more (e.g., 1,000, 1,500, 2,000, 2,500, 5,000, 7,500, 10,000 nM or more). In some embodiments, the disclosed compounds have a LD₅₀ in human foreskin fibroblast cells of greater than 10,000 nM. Illustrative LD₅₀ measurements are described herein at the Examples.

In some embodiments, the disclosure provides a method of treating a tumor expressing aberrant CD19 levels (e.g., breast cancer, or more specifically, triple negative breast cancer) in a patient comprising administering to the patient a therapeutically effective amount of a compound disclosed herein. In some embodiments, the treatment results in an at least 10% reduction in tumor volume. In some cases, the reduction in tumor volume is at least 20%, at least 25%, at least 30%, at least 40%, or at least 50%. The reduction can occur within 12 months of initiating therapy, within 11 months, within 10 months, within 9 months, within 8 months, within 7 months, within 6 months, within 5 months, within 4 months, within 3 months, within 2 months, or within 1 month of initiating therapy.

As used herein, the term “patient” (e.g., subject) refers to an animal, specifically a “mammal” including a non-primate (e.g., a cow, pig, horse, sheep, rabbit, guinea pig, rat, cat, dog, or mouse) and a primate (e.g., a monkey, chimpanzee, or human), and more specifically a human. In a preferred embodiment, the patient is a “human”.

As used herein, an “effective amount” refers to an amount sufficient to elicit the desired biological response. As used herein, a “safe and effective amount” of a compound or composition described herein is an effective amount of the compound or composition which does not cause excessive or deleterious side effects in a patient.

As described in the Examples, when cells from a TNBC cell line (e.g., MDA-MB-231) are treated with a compound as disclosed herein, the treated cells undergo cell cycle arrest and apoptosis. In contrast, when a normal human fibroblast cell line (e.g., human foreskin fibroblast 2088 cells) are treated in the same manner, the treated cells continue to proliferate in a healthy manner. Thus, the compounds disclosed herein can selectively target a cancer cell in the presence of a healthy cell, which indicates a likelihood that healthy cells are not impacted, or minimally impacted, by the compound.

In some embodiments, the disclosure provides a method of treating cancer in patient comprising administering to the patient a therapeutically effective amount of the compound, tautomer, or salt of the disclosure. In some embodiments, the cancer is breast cancer, prostate cancer, cancer of the gastrointestinal tract (e.g., colorectal cancer), bladder cancer, sarcoma, cervical cancer, esophageal adenocarcinoma, acute myeloid leukemia, melanoma, glioma, or ovarian cancer. In some embodiments, the cancer is breast cancer. In some embodiments, the disclosure provides a method of treating a patient having triple negative breast cancer comprising administering a therapeutically effective dose of a compound or pharmaceutical composition to treat the triple negative breast cancer.

### Combination Therapy

A compound described herein, or a pharmaceutically acceptable salt thereof, can be administered alone or in combination with an additional suitable therapy, for example, a second therapeutic agent, such as an anticancer agent.

Thus, in some embodiments, the patient undergoes one or more additional therapies in addition to treatment with a compound as disclosed herein.

When combination therapy is employed, a safe and effective amount can be achieved using a first amount of a compound as disclosed herein, or a pharmaceutically acceptable salt thereof, and a second amount of an additional suitable therapeutic agent (e.g. an anticancer agent).

In some embodiments, the second therapy is selected from chemotherapy (e.g., a chemotherapeutic), radiation therapy, immunotherapy (e.g., an immunotherapeutic), surgery, and a combination thereof. In some embodiments, the second therapy comprises surgery to remove breast tissue.

### Embodiments

1. A compound, tautomer, or pharmaceutically acceptable salt thereof, having a structure of formula (I):

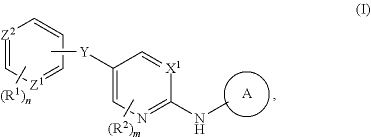

wherein:
X¹ is CH, CR², or N;
Y is selected from the group consisting of a bond, CR^{a}R^{b}, NRC, C(O), O, S, SO₂, C(O)NH, and HNC(O);
each of Z¹ and Z² is independently CH, CR¹, or N;
each of R^{a} and R^{b} is independently H, C₁-C₆alkyl, hydroxy, or halo, or R^{a} and R^{b} taken together with the carbon atom which they are attached form a spiro C₃-C₆cycloalkyl;
R^{c} is H or C₁-C₆alkyl;
ring A comprises a C₆-C₁₀aryl, a C₃-C₆ cycloalkyl, or a 6-membered cycloheteroalkyl comprising 1, 2, or 3 ring heteroatoms independently selected from N, O, and S, wherein ring A is optionally substituted with 1-3 substituents independently selected from the group consisting of halo, hydroxy, —CN, C₁-C₆alkyl, C₁-C₆haloalkyl, C₃-C₆cycloalkyl, a spiro C₃-C₆ cycloalkyl, C₁-C₆alkoxy, C₁-C₆haloalkoxy, C₃-C₆cycloalkoxy, C₃-C₆cycloalkyl-C₁-C₆alkylene, C₆-C₁₀aryl, C₅-C₁₀cycloalkyl, 5-10 membered cycloheteroalkyl comprising 1, 2, or 3 ring heteroatoms independently selected from N, O, and S, NR′R″, and C(O)NR′R″;
n is 0, 1, or 2;
each R¹ is independently selected from the group consisting of halo, hydroxy, cyano, C₁-C₆alkyl, C₁-C₆haloalkyl, C₃-C₆cycloalkyl, C₁-C₆alkoxy, C₁-C₆haloalkoxy, C₂-4alkynylene-phenyl, C₃-C₆cycloalkoxy optionally substituted with C₁-C₆alkyl, C₅-C₆heteroaryl comprising 1, 2, or 3 ring heteroatoms independently selected from N, O, and S NR′R″, C(O)NR′R″, and 6-10 membered cycloheteroalkoxy comprising 1, 2, or 3 ring heteroatoms independently selected from N, O, and S, and the cycloalkyl, cycloalkoxy, phenyl, heteroaryl, and cycloheteroalkoxy ring is substituted with 0, 1, or 2 substituents independently selected from C₁₋₆alkyl, halo, C₁₋₆alkoxy, C₁₋₆haloalkyl, C₁₋₆haloalkoxy, and C₃₋₆cycloalkyl; or
when two R¹ are ortho to each other, taken together with the atoms to which they are attached they form a fused 5 or 6 membered aromatic ring comprising 0-3 ring heteroatoms independently selected from N, O, and S, and is optionally substituted with 1-2 substituents independently selected from C₁-C₆alkyl and oxo;
m is 0, 1, or 2;
each R² is independently C₁-C₆ alkyl;
each R′ and R″ is independently selected from the group consisting of H, C₁-C₁₀alkyl, and C₃-C₆cycloalkyl; or taken together with the nitrogen to which they are attached form a 4-8 membered heterocycle including 0-2 additional ring heteroatoms independently selected from N, O, and S;
with the proviso that when X¹ is N, m is 0, Z¹ is N, Y is para to Z¹, Z² is CH, ring A is phenyl optionally substituted with NH₂ or CH₃, n is 0 or 2, and each R¹ is NH₂, then Y is not a bond.
2. The compound, tautomer, or salt of embodiment 1, wherein each of Z¹ and Z² is independently CH or N.
3. The compound, tautomer, or salt of embodiment 1 or 2, wherein Z¹ and Z² are each CH.
4. The compound, tautomer, or salt of embodiment 1 or 2, wherein Z¹ and Z² are each N.
5. The compound, tautomer, or salt of embodiment 1 or 2, wherein Z¹ is N and Z² is CH.
6. The compound, tautomer, or salt of embodiment 1 or 2, wherein Z¹ is NH and Z² is CH.
7 The compound, tautomer, or salt of embodiment 5, wherein the structure of formula (I) is a structure selected from one of formulae (IA)-(IG):

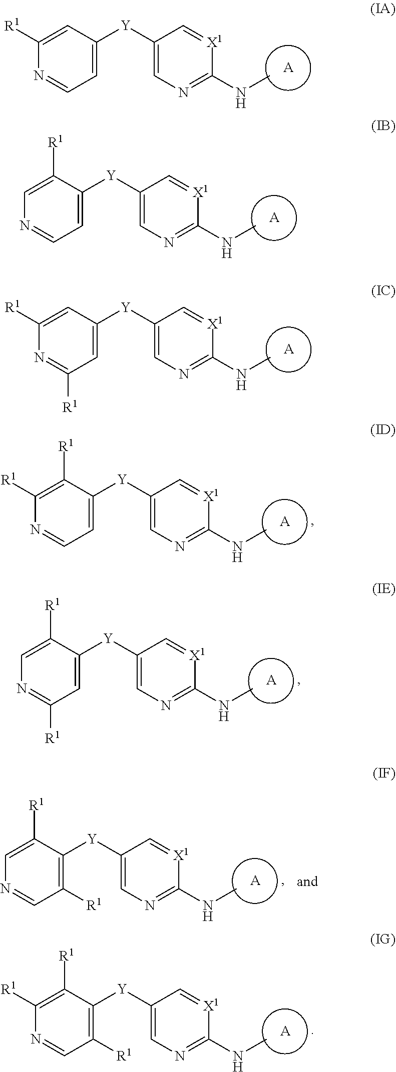

8. The compound, tautomer, or salt of embodiment 7 having a structure of formula (IA):

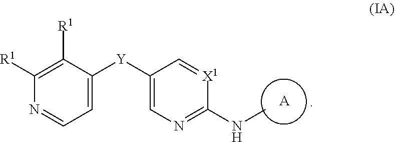

9. The compound, tautomer, or salt of any one of embodiments 1-8, wherein n is 0.
10. The compound, tautomer, or salt of any one of embodiments 1-8, wherein n is 1.
11. The compound, tautomer, or salt of any one of embodiments 1-8, wherein n is 2.
12. The compound, tautomer, or salt of any one of embodiments 1-11, wherein each R¹ is independently selected from the group consisting of halo, hydroxy, cyano, C₁-C₆alkyl, C₁-C₆haloalkyl, C₃-C₆cycloalkyl, C₁-C₆alkoxy, C₁-C₆haloalkoxy, C₃-C₆cycloalkoxy, NR′R″, C(O)NR′R″, and 6-10 membered cycloheteroalkoxy comprising 1, 2, or 3 ring heteroatoms independently selected from N, O, and S.
13. The compound, tautomer, or salt of any one of embodiments 1-8, and 10-12, wherein each R¹ is selected from the group consisting of halo, C₁-C₆alkyl, C₁-C₆alkoxy, C₁-C₆haloalkoxy, C₃-C₆cycloalkyl, C₃-C₆cycloalkoxy, and C(O)NR′R″.
14. The compound, tautomer, or salt of embodiment 13 wherein each R¹ is independently selected from the group consisting F, Cl, Br, I, methyl, ethyl, isopropyl, cyclopropyl, butyl, cyclobutyl, pentyl, cyclopentyl, cyclohexyl, hydroxy, keto, methoxy, fluoromethoxy, difluoromethoxy, trifluoromethoxy, ethoxy, propoxy, isopropoxy, butoxy, cyclobutoxy, pentoxy, cyclopentoxy, hexoxy, cyclohexoxy, pyridinyl, 1-naphthyl, 2-naphthyl, —C(O)NR′R″, methylNH—, ethylNH—, isopropylNH—, cyclopropylNH—, butylNH—, cyclobutylNH—, pentylNH—, cyclopentylNH—, hexylNH—, cyclohexylNH—, heptylNH—, and 6-10 membered cycloheteroalkoxy.
15. The compound, tautomer, or salt of any one of embodiments 1-11, wherein at least one R¹ is hydrogen, cyclopentoxy, methyl, methoxy, isopropyl, trifluoromethyl, —C(O)NHMe, N-methylacetamido, ethyl, cyclohexoxy, piperidin-3-yl-O—, fluoro, chloro, trifluoromethoxy, cyclopropyl, cyclopentylNH—, cyano, oxo, cyclopentyl-NHC(O)—, —CH₂—NH—C(O)—,

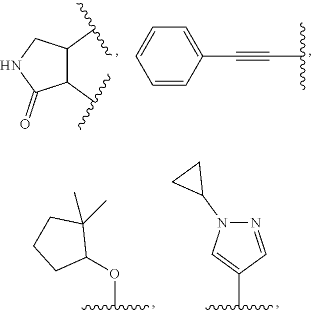

or amino, or two ortho R¹ together form

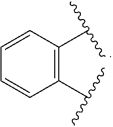 [SMILES: *c1ccccc1*]

16. The compound, tautomer, or salt of embodiment 13, wherein R¹ is C₃-C₆cycloalkoxy.
17. The compound, tautomer, or salt of embodiment 16, wherein R¹ is cyclopentoxy.
18. The compound, tautomer, or salt of embodiment 13, wherein R¹ is C₁-C₆alkyl.
19. The compound, tautomer, or salt of embodiment 18, wherein R¹ is methyl.
20 The compound, tautomer, or salt of any one of embodiments 1-8 and 10-19, wherein two R¹ are ortho to each other and taken together with the atoms to which they are attached form a 6 membered aryl.
21. The compound, tautomer, or salt of embodiment 20, wherein the 6 membered aryl is substituted with methyl.
22. The compound, tautomer, or salt of any one of embodiments 1-21, wherein R′ is H.
23. The compound, tautomer, or salt of any one of embodiments 1-22, wherein R″ is C₁-C₆alkyl.
24 The compound, tautomer, or salt of embodiment 23, wherein R″ is methyl.
25. The compound, tautomer, or salt of any one of embodiments 1-24, wherein ring A is optionally substituted with 1, 2, or 3 substituents selected from the group consisting of F, Cl, Br, I, hydroxy, NH₂, NHR′, methyl, ethyl, propyl, cyclopropyl, butyl, cyclobutyl, isobutyl, tert-butyl, pentyl, cyclopentyl, hexyl, cyclohexyl, methoxy, fluoromethoxy, difluoromethoxy, trifluoromethoxy, ethoxy, 1,1,2,2-tetrafluoroethoxy, perfluoroethoxy, propoxy, isopropoxy, cyclopropoxy, butoxy, cyclobutoxy, isobutoxy, tert-butoxy, pentoxy, cyclopentoxy, hexoxy, cyclohexoxy, cyclopropylmethyl, cyclobutylmethyl, piperazinyl, morpholinyl, 1-naphthyl, 2-naphthyl, tetrahydronapthyl, and isocromenyl.
26. The compound, tautomer, or salt of any one embodiments 1-25, wherein ring A is selected from the group consisting of phenyl, cyclohexyl, 4-piperidinyl, and tetrahydropyranyl, wherein ring A is optionally substituted.
27. The compound, tautomer, or salt of embodiment 1-26, wherein ring A is cyclohexyl optionally substituted.
28. The compound, tautomer, or salt of embodiment 27 wherein ring A is cyclohexyl substituted at the 4-position with a substituent selected from the group consisting of methyl, methoxy, and isopropoxy.
29. The compound, tautomer, or salt of embodiment 1-26, wherein ring A is 4-piperidinyl optionally substituted.
30. The compound, tautomer, or salt of embodiment 29, wherein ring A is 4-piperidinyl substituted on ring N with methyl or isobutyl.
31. The compound, tautomer, or salt of embodiment 1-26, wherein ring A is tetrahydropyranyl optionally substituted.
32. The compound, tautomer, or salt of any one of embodiments 1-26, wherein ring A is phenyl optionally substituted.
33. The compound, tautomer, or salt of embodiment 32, wherein ring A is phenyl substituted at the 2-position with a substituent selected from the group consisting of F, Cl, Br, I, C₁-C₆alkoxy, hydroxy, NH₂, and NHR′.
34. The compound, tautomer, or salt of embodiment 32, wherein ring A is phenyl substituted at the 3-position or 4-position with a substituent selected from the group consisting of F, Cl, Br, I, C₁-C₆alkyl, C₁-C₆alkoxy, C₁-C₆-haloalkyl, C₃-C₆cycloalkyl, C₃-C₆cycloalkoxy, C₃-C₆cycloalkyl-C₁-C₆alkylene, C₆-C₁₀aryl, C₆-C₁₀cycloalkyl, and 6-10 membered heterocycloalkyl comprising 1, 2, or 3 ring heteroatoms independently selected from N, O, and S.
35. The compound, tautomer, or salt of embodiment 32, wherein ring A is phenyl substituted at the 4-position with a substituent selected from the group consisting of F, Cl, Br, I, C₁-C₆alkyl, C₁-C₆alkoxy, C₃-C₆cycloalkyl, hydroxy, NH₂, and NHR′.
36. The compound, tautomer, or salt of any one of embodiments 1-24, wherein ring A is 4-isobutylphenyl.
37. The compound, tautomer, or salt of any one of embodiments 1-24, wherein ring A is 4-cyclobutylphenyl.
38. The compound, tautomer, or salt of any one of embodiments 1-37, wherein X¹ is CH or N.
39. The compound, tautomer, or salt of any one of embodiments 1-37, wherein X¹ is N and m is 0.
40. The compound, tautomer, or salt of any one of embodiments 1-37, wherein X¹ is N, m is 1, and R² is methyl.
41. The compound, tautomer, or salt of any one of embodiments 1-37, wherein X¹ is CH and m is 0.
42. The compound, tautomer, or salt of any one of embodiments 1-41, wherein Y is NH.
43. The compound, tautomer, or salt of any one of embodiments 1-41, wherein Y is a bond.
44. The compound, tautomer, or salt of any one of embodiments 1-41, wherein Y is NHC(O) or C(O)NH.
45. The compound, tautomer, or salt of any one of embodiments 1-41, wherein Y is CH₂.
46. The compound, tautomer, or salt of any one of embodiments 1-41, wherein Y is C(O).
47. The compound, tautomer, or salt of any one of embodiments 1-46, wherein each of R^{a} and R^{b} is independently H or C₁-C₆alkyl.
48. The compound, tautomer, or salt of embodiment 1 having a structure as recited in Table A.
49. A pharmaceutical composition comprising the compound, tautomer, or salt of any one of embodiments 1-48 and a pharmaceutically acceptable excipient.
50. A method of inhibiting cyclin dependent kinase 19 (CDK19) comprising contacting CDK19 with the compound, tautomer, or salt of any one of embodiments 1-48 in an amount effective to inhibit CDK19.
51. The method of embodiment 50, wherein the compound inhibits CDK19 selectively over cyclin dependent kinase 8 (CDK8).
52. The method of embodiment 51, wherein the compound is at least 2 times more selective for CDK19 over CDK8.
53. The method of embodiment 52, wherein the compound is at least 3 times more selective for CDK19 over CDK8.
54. The method of embodiment 53, wherein the compound is at least 20 times more selective for CDK19 over CDK8.
55. The method of any one of embodiments 50-54, wherein the compound has an IC₅₀ for CDK19 of less than 400 nM.
56. The method of embodiment 55, wherein the compound has an IC₅₀ for CDK19 of less than 200 nM.
57. A method of treating cancer in a patient comprising administering to the patient a therapeutically effective amount of the compound, tautomer, or salt of any one of embodiments 1-48.
58. The method of embodiment 57, wherein the cancer is breast cancer, prostate cancer, cancer of the gastrointestinal tract (e.g., colorectal cancer), bladder cancer, sarcoma, cervical cancer, esophageal adenocarcinoma, acute myeloid leukemia, melanoma, glioma, or ovarian cancer.
59. The method of embodiment 57, wherein the cancer is breast cancer.
60. The method of embodiment 59, wherein the breast cancer is triple negative breast cancer.
61. The method of embodiment 59 or 60, further comprising removing breast tissue from the patient.
62. The method of any one of embodiments 57-61, further comprising administering a second therapeutic agent to the patient.
63. The method of embodiment 62, wherein the second therapeutic agent comprises radiation, an immunotherapeutic, or a chemotherapeutic.

### EXAMPLES

The following examples further illustrate the disclosed tablet formulation and process, but of course, should not be construed as in any way limiting its scope.

The following abbreviations are used in the Examples: TLC refers to thin layer chromatography; UV refers to ultraviolet; ACS refers to American Chemical Society; ESI refers to electrospray ionization; MS refers to mass spectrometry; LC refers to liquid chromatography; FRET refers to Förster resonant energy transfer.

General Chemical Methods: All the reagents obtained from commercial suppliers were used as received. Reactions were performed under ambient atmosphere unless otherwise noted. Precoated Merck F-254 silica gel plates were used for thin layer analytical chromatography (TLC) and visualized with short wave UV light. Purification was performed on a Biotage Selekt purification system using silica gel flash cartridges (Biotage Sfar Silica D, Duo 60 μm). Proton magnetic resonance (¹H NMR) spectra were recorded on a Bruker AV-500 spectrometer unless otherwise mentioned. Chemical shifts (δ) are expressed in parts per million relatives to residual CHCl₃, MeOH or DMSO as internal standards. Abbreviations are: s, singlet; d, doublet; t, triplet; q, quartet; p, quintet; sex, sextet; sept, septet; app, apparent. Low-resolution mass spectra were collected on Agilent 1260 Infinity II single quad LCMS instrument.

### General Procedure for the Synthesis of Amines as Exemplified by the Synthesis of N2-(4-isobutylphenyl)-N5-(pyridin-3-yl)pyrimidine-2,5-diamine (Compound A52)

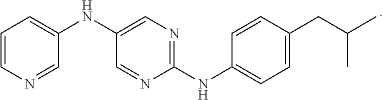 [SMILES: CC(C)Cc1ccc(Nc2ncc(Nc3cccnc3)cn2)cc1]

Synthesis of 1-isobutyl-4-nitrobenzene. A round-bottomed flask containing a magnetic stir bar was charged with 1-bromo-4-nitro-benzene (10 g, 1 equiv.), isobutylboronic acid (6.5 g, 1.3 equiv.), tricyclohexyl phosphine (1.4 g, 0.1 equiv.), and flushed with nitrogen. To this mixture was added, degassed toluene (150 mL) and water (10 mL). To the reaction mixture was added K₃PO₄ (31.5 g, 3 equiv.), Pd(OAc)₂ (555 mg, 0.05 equiv.) and the mixture was heated at 100° C. for 3 h. Upon completion, diluted with water and extracted with EtOAc (3×). The organic layer was washed with brine and dried over sodium sulfate and concentrated. The crude residue obtained was further purified using flash column (0-10% EtOAc in hexenes).

Synthesis of 4-isobutylaniline. A round-bottomed flask containing a magnetic stir bar was charged with 1-isobutyl-4-nitro-benzene (8.5 g, 1 equiv.), EtOH (150 mL), Fe (8 g, 3 equiv.) and AcOH (47 mL). The reaction mixture was heated at 100° C. for 3 h. After completion, reaction was neutralized with sat. NaHCO₃ solution and filtered to remove solid impurities. Filtrated was extracted with EtOAc (3×). The organic layer was washed with brine solution, dried over sodium sulfate and concentrated to rotary evaporator. The crude mixture obtained was further purified using column chromatography. LCMS (ESI) m/z [M+H]+150.10.

Synthesis of N-(4-isobutylphenyl)-5-nitropyrimidin-2-amine. A round bottomed flask containing a magnetic stir bar was charged with Cs₂CO₃ (3.06 g, 1.5 equiv.) and dried over high vacuum. To this mixture was added 2-chloro-5-nitro-pyrimidine (1 g, 1 equiv.), 4-isobutylaniline (935 mg, 1 equiv.), 2-Me-THF (60 mL) under nitrogen atmosphere. The reaction mixture was heated at 100° C. for 3 h. LC-MS showed the completion. Reaction was brought to room temperature, neutralized with 1M HCl and extracted with EtOAc. The organic layer was washed with brine solution, dried over sodium sulfate and concentrated. The crude product obtained was used without further purification in the next step. LCMS (ESI) m/z [M+H]+273.00.

Synthesis of N2-(4-isobutylphenyl)pyrimidine-2,5-diamine. A round bottomed flask containing a magnetic stir bar was charged with N-(4-isobutylphenyl)-5-nitropyrimidin-2-amine (1.68 g, 1 equiv.) EtOH (50 mL), acetic acid (6 mL) and Fe (1.04 g, 3 equiv.). The reaction mixture was heated at 100° C. for 3 h. After completion, the reaction was neutralized with sat. NaHCO₃ solution and filtered off to remove solid impurities. The filtrate was extracted with EtOAc (3×). The organic layer was washed with brine solution, dried over sodium sulfate and concentrated to rotary evaporator. The crude mixture obtained was further purified using column chromatography. LCMS (ESI) m/z [M+H]+243.10.

Synthesis of Compound A52. A round bottomed flask containing a magnetic stir bar was charged with N2-(4-isobutylphenyl)pyrimidine-2,5-diamine (100 mg, 1 equiv.), 3-chloropyridine (46 mg, 1 equiv.), K₃PO₄ (122 mg, 1.4 equiv.), tBuXPhos Pd G3 (10 mg, 3 mol %) and brought under the nitrogen atmosphere. To this mixture was added, anhydrous 1,4-dioxane (1 mL) and the reaction mixture was heated at 120° C. for 3 h. After completion, reaction was diluted with water and extracted with EtOAc (3×). The organic layer was washed with brine solution, dried over sodium sulfate and concentrated. The crude mixture obtained was further purified using column chromatography (0-10% MeOH in EtOAc. 1H NMR (500 MHZ, DMSO-d6) δ9.43 (s, 1H), 8.38 (s, 2H), 8.19 (d, J=2.7 Hz, 1H), 8.03 (s, 1H), 7.95 (dd, J=4.4, 1.4 Hz, 1H), 7.63 (d, J=8.5 Hz, 2H), 7.29-7.13 (m, 2H), 7.05 (d, J=8.5 Hz, 2H), 2.38 (d, J=7.2 Hz, 2H), 1.79 (hept, J=6.8 Hz, 1H), 0.86 (d, J=6.6 Hz, 6H). LCMS (ESI) m/z [M+H]+320.20.

### General Procedure for the Synthesis of Ketones as Exemplified by the Synthesis of (2-((4-isobutylphenyl)amino) pyrimidin-5-yl) (3-methylpyridin-4-yl) methanone (Compound A73)

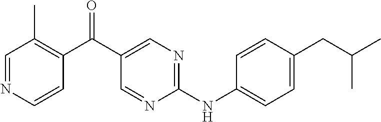 [SMILES: Cc1cnccc1C(=O)c1cnc(Nc2ccc(CC(C)C)cc2)nc1]

Synthesis of N-methoxy-N,3-dimethylisonicotinamide. A round bottomed flask containing a magnetic stir bar was charged with 3-methylpyridine-4-carboxylic acid (250 mg, 1 equiv.), DCE (5 mL), DMF (0.001 mL, 0.004 equiv.) at room temperature under nitrogen atmosphere. To this was added oxalyl chloride (0.2 mL, 1.25 equivalent). After stirring for 1.5 hours, the solvent was removed and the excess oxalyl chloride allowed to evaporate. To the crude mixture was added DCM (5 mL), N-methoxymethanamine hydrochloride (245 mg, 1.4 equivalent), and NEt₃ (0.76 mL, 3 equivalent) at room temperature. After 1 hours, the reaction was quenched with a saturated solution of NaHCO₃ and extracted the mixture twice with DCM. The organic layer was washed with brine, dried over sodium sulfate, and concentrated. Further drying over high vacuum resulted in product. LCMS (ESI) m/z [M+H]+181.00.

Synthesis of 5-bromo-N-(4-isobutylphenyl)pyrimidin-2-amine. A round bottomed flask containing magnetic stir bar was charged with 5-bromo-2-chloro-pyrimidine (1 g, 1 equiv.), 4-isobutylaniline (771 mg, 1 equiv.), t-BuOH (15 mL) and DIPEA (1.07 mL, 1.2 equiv.). The reaction mixture was heated at 100° C. for 20 h and the brought to room temperature. The reaction mixture was then diluted with water and extracted with EtOAc (3×). The organic layer washed with brine, dried over sodium sulfate and the solvent was removed using rotary evaporator. The crude mixture obtained was further purified using column chromatography. LCMS (ESI) m/z [M+H] 306.00.

Synthesis of Compound A73. To an oven dried round bottom flask was added a solution of 5-bromo-N-(4-isobutylphenyl)pyrimidin-2-amine (68 mg), in anhydrous 2-Me-THF (4 mL) under inert atmosphere and then the reaction mixture was cooled to −78° C. A solution of n-BuLi (0.12 mL) in anhydrous hexane was added to the reaction mixture at −78° C. Subsequently, a solution of N-methoxy-N, 3-dimethyl-pyridine-4-carboxamide (40 mg) in anhydrous THF was added to the mixture at −78° C. The reaction was quenched by addition of water, extracted with ethyl acetate for three times. The organic extracts were dried over Na₂SO₄ and concentrated in vacuum. The product obtained was purified by a column chromatography (40% EtAOc/Hexenes) to give the desired ketone. ¹H NMR (500 MHz, methanol-d4) δ8.72 (s, 2H), 8.58 (s, 1H), 8.53 (d, J=5.0 Hz, 1H), 7.59 (d, J=8.5 Hz, 2H), 7.38 (d, J=5.0 Hz, 1H), 7.12 (d, J=8.5 Hz, 2H), 2.46 (d, J=7.2 Hz, 2H), 2.32 (s, 3H), 1.85 (dp, J=13.6, 6.8 Hz, 1H), 0.91 (d, J=6.7 Hz, 6H). LCMS (ESI) m/z [M+H]+347.20.

### General Procedure for the Synthesis of Amides as Exemplified by the Synthesis of N-(2-((4-isobutylphenyl)amino) pyrimidin-5-yl) isonicotinamide (Compound A48)

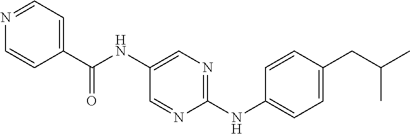 [SMILES: CC(C)Cc1ccc(Nc2ncc(NC(=O)c3ccncc3)cn2)cc1]

A round bottomed flask containing stir bar was charged with N2-(4-isobutylphenyl)pyrimidine-2,5-diamine (34 mg, 1 equiv.), DCM (1 mL) and NEt₃ (0.03 mL). To this mixture was added, pyridine-4-carbonyl chloride (20 mg, 1 equiv.) and stirred at room temperature for 1 h. The crude product obtained was purified using column chromatography (0-5% MeOH in EtOAc). ¹H NMR (500 MHZ, DMSO-d6) δ10.57 (s, 1H), 9.57 (s, 1H), 8.82-8.80 (m, 2H), 8.77 (s, 2H), 8.02-7.79 (m, 2H), 7.63 (d, J=8.5 Hz, 2H), 7.07 (d, J=8.6 Hz, 2H), 2.39 (d, J=7.1 Hz, 2H), 1.80 (hept, J=6.7 Hz, 1H), 0.86 (d, J=6.6 Hz, 6H). LCMS (ESI) m/z [M+H]+348.20.

### General Procedure for Synthesis of Reverse Amides as Exemplified by the Synthesis of 2-((4-isobutylphenyl)amino)-N-(pyridin-4-yl)pyrimidine-5-carboxamide (Compound A49)

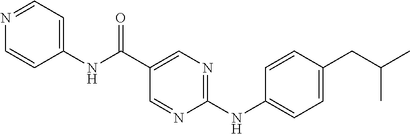 [SMILES: CC(C)Cc1ccc(Nc2ncc(C(=O)Nc3ccncc3)cn2)cc1]

Synthesis of 2-((4-isobutylphenyl)amino) pyrimidine-5-carboxylic acid. A round bottomed flask containing a stir bar was charged with Cs₂CO₃ (1.13 g, 1.5 equiv.) and dried over high vacuum and flush with nitrogen. To this mixture was added, methyl 2-chloropyrimidine-5-carboxylate (400 mg, 1 equiv.), 4-isobutylaniline (346 mg, 1 equiv.) and 2-Me-THF (20 mL). The reaction mixture was heated at 100° C. for 3 h and completion was seen in LCMS. The reaction mixture was neutralized with 1 M HCl and extracted with EtOAc (3×). The organic layer was washed with brine and dried over sodium sulfate and concentrated. To the crude mixture obtained was added EtOH (10 mL) and 2 M NaOH (2.5 mL, 2 equiv.) and this mixture was heated at 80° C. for 1 h. The reaction mixture was concentrated to remove ethanol and acidified with 1 M HCl to precipitate the product.

Synthesis of Compound A49. A round-bottomed flask containing a magnetic stir bar was charged with 2-(4-isobutylanilino)pyrimidine-5-carboxylic acid (100 mg, 1 equiv.) and flushed with nitrogen. To this was added, DCE (5 mL), DMF (0.001 mL, 0.004 equivalent) and oxalyl chloride (0.04 mL, 1.25 equivalent) at room temperature. After stirring for 1.5 h, the solvent and excess oxalyl chloride were removed by evaporation. To the residue was added DCM (5 mL), pyridin-4-amine (42 mg, 1.2 equiv.), and NEt₃ (0.15 mL, 3 equiv.) at room temperature. After 2 hours, the reaction was quenched with water and the mixture extracted with DCM (2×). The organic layer was washed with brine and dried over sodium sulfate and concentrated. The crude residue obtained was further purified using column chromatography (0-5% MeOH in EtOAc). ¹H NMR (500 MHZ, chloroform-d) δ8.91 (s, 2H), 8.70-8.51 (m, 2H), 7.80 (s, 1H), 7.65-7.58 (m, 2H), 7.53 (d, J=8.4 Hz, 2H), 7.45 (s, 1H), 7.16 (d, J=8.4 Hz, 2H), 2.47 (d, J=7.1 Hz, 2H), 1.87 (dp, J=13.5, 6.7 Hz, 1H), 0.92 (d, J=6.6 Hz, 6H). LCMS (ESI) m/z [M+H]+348.30.

### General Procedure for the Synthesis Aryl-Pyrimidines as Exemplified by the Synthesis of 5-(2,6-dimethylpyridin-4-yl)-N-(4-isobutylphenyl)pyrimidin-2-amine (Compound A101)

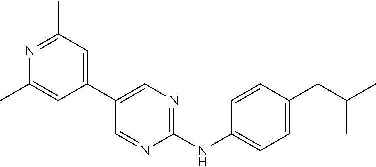 [SMILES: Cc1cc(-c2cnc(Nc3ccc(CC(C)C)cc3)nc2)cc(C)n1]

A round-bottomed flask containing a magnetic stir bar was charged with 5-bromo-N-(4-isobutylphenyl)pyrimidin-2-amine (100 mg, 1 equiv.), 4-pyridylboronic acid (54 mg, 1.1 equiv.), PCy3 (24 mg, 0.2 equiv.), Pd (dppf) Cl₂ (23 mg, 0.1 equiv.), K₃PO₄ (115 mg, 1.7 equiv.) and brought under nitrogen. To this mixture was added, 1,4-dioxane (3 mL) and water (0.6 mL) and heated at 100° C. for 3 h. The reaction mixture was diluted with water and extracted with EtOAc (3×). The organic layer was washed with brine and dried over sodium sulfate and concentrated. The crude residue obtained was further purified using column chromatography (50% EtOAc/hexenes). LCMS (ESI) m/z [M+H]+332.20.

Compounds as disclosed herein were prepared in a similar manner as described in detail above.

### Biological Methods

FRET Displacement Assay to Measure IC₅₀: The CDK19/CDK8 IC₅₀ values were measured to evaluate activity and to determine CDK19/CDK8 selectivity. The IC₅₀ values of the disclosed compounds was measured using a LanthaScreen™ europium kinase binding assay (ThermoFisher), as described herein.

To a kinase buffer cocktail solution (i.e., 5× Kinase Buffer A) was added: purified recombinant his-tagged CDK19/CycC protein (5 nM), ATP-competitive kinase inhibitor scaffold kinase tracer Alexa Fluor® 665 (10 nM), biotin anti-his tag antibody (2 nM), LanthaScreen® europium-streptavidin (2 nM). An aliquot of the cocktail solution (10 μL) was added to each well of a LUMITRAC™ 200:384 flat bottom, non-treated microtiter white plate. The plate was then covered to protect light sensitive reagents and incubated for 30 min at room temperature to equilibrate before addition of any inhibitors.

A serial dilution of the compounds was titrated into each well using an automated liquid handling Staccato integrated system at the following concentration(s) (Log₁₀ Molar [C]): −4.5, −5.0, −5.5, −6.0, −6.5, −7.0, −7.5, −8.0 so that a complete dose-response could be calculated. A Tecan microplate reader infinite m200 instrument was then used to measure the FRET signal of each individual well using an excitation of 317/20 nm, emission europium of 620/12 nm, and emission kinase tracer of 665/12 nm after a 3 h incubation period at room temperature until steady-state kinetics were achieved.

Results were performed in duplicate and were repeated a total of 3 independent times before data was normalized to the DMSO control group and IC₅₀ values reported. The results are summarized in Table 1.

TABLE 1
IC₅₀ and CDK19/CDK8 Selectivity

| | CDK19 | CDK8 | |
|---|---|---|---|
| | IC₅₀ | IC₅₀ | CDK19 |
| ID | (nM) | (nM) | Selectivity |
| A1 | 69 | 59 | 1.2 |
| A2 | 11 | 14 | 1.2 |
| A3 | 7.6 | 8.2 | 1.1 |
| A5 | 62 | 186 | 3 |
| A6 | 38 | 210 | 5.5 |
| A7 | 10000 | 14000 | — |
| A8 | 231 | 767 | 3.3 |
| A9 | 2070 | 8260 | 4 |
| A10 | 99 | 310 | 3.1 |
| A11 | 25 | 22 | 0.9 |
| A12 | 38 | 34 | 0.9 |
| A13 | 24 | 56 | 2.4 |
| A14 | 29 | 96 | 3.3 |
| A15 | 96 | 109 | 1.1 |
| A16 | 133 | 371 | 2.8 |
| A17 | 419 | 1160 | 2.8 |
| A18 | 10000 | 10000 | — |
| A19 | 9.1 | 38 | 4.2 |
| A20 | 10000 | 10000 | — |
| A21 | 38 | 564 | 15 |
| A22 | 24 | 31 | 1.3 |
| A23 | 100 | 451 | 4.5 |
| A24 | 9870 | 10000 | — |
| A25 | 16 | 15 | 0.9 |
| A26 | 5690 | 9030 | 1.6 |
| A27 | 10000 | 10000 | — |
| A28 | 1640 | 2730 | 1.7 |
| A29 | 991 | 2030 | 2 |
| A30 | 10000 | 10000 | — |
| A31 | 9030 | 9930 | — |
| A32 | 378 | 1320 | 3.5 |
| A33 | 965 | 2870 | 3 |
| A34 | 903 | 4510 | 5 |
| A35 | 10000 | 10000 | — |
| A36 | 123 | 513 | 4.2 |
| A37 | 41 | 83 | 2 |
| A38 | 10000 | 10000 | — |
| A39 | 9.5 | 19 | 2 |
| A40 | 25000 | | |
| A41 | 8940 | 8944 | 1 |
| A42 | 449 | 833 | 2 |
| A43 | 6 | 37 | 6 |
| A44 | 4814 | 6334 | 1 |
| A45 | 138 | 214 | 2 |
| A46 | 88 | 407 | 4.6 |
| A47 | 308 | 737 | 2.4 |
| A48 | 1550 | 8220 | 5.3 |
| A49 | 2490 | 9360 | 3.9 |
| A50 | ≤2.5 | 16 | ≥6 |
| A51 | ≤2.5 | 38 | ≥15 |
| A52 | 704 | 3550 | 5 |
| A53 | ≤2.5 | 56 | ≥22 |
| A54 | 45 | 35 | 0.8 |
| A55 | 40 | 44 | 1.1 |
| A56 | ≤2.5 | 27 | ≥11 |
| A57 | ≤2.5 | 3 | 2 |
| A58 | ≤2.5 | 2.8 | 2 |
| A59 | 11 | 23 | 2 |
| A61 | 7.6 | 7 | 1 |
| A62 | ≤2.5 | 3 | 1.5 |
| A63 | 1600 | 7790 | 4.8 |
| A64 | 389 | 905 | 2.3 |
| A65 | 1160 | 2270 | 2 |
| A66 | 4.6 | 5.8 | 1.2 |
| A67 | 106 | 106 | 1 |
| A68 | 12 | 12 | 1 |
| A69 | 1410 | 2670 | 1.9 |
| A70 | ≤2.5 | 46 | ≥18 |
| A71 | ≤2.5 | 6 | 3 |
| A72 | ≤2.5 | 34 | ≥14 |
| A73 | ≤2.5 | 16 | ≥6 |
| A75 | 296 | 1160 | 3.9 |
| A76 | 1720 | 5100 | 3 |
| A77 | 1080 | 3760 | 3.5 |
| A78 | 5 | 13 | 2.6 |
| A79 | 10000 | 10000 | — |
| A80 | 3330 | 10000 | 3 |
| A81 | 10000 | 10000 | — |
| A82 | 131 | 193 | 1.5 |
| A83 | 5900 | 8300 | 1 |
| A84 | 10000 | 10000 | — |
| A85 | 72 | 268 | 3.8 |
| A86 | 1110 | 10000 | 9 |
| A87 | 84 | 198 | 2.4 |
| A88 | 557 | 1490 | 2.7 |
| A89 | 10000 | 10000 | — |
| A90 | 1060 | 4860 | 4.6 |
| A91 | 798 | 10000 | 12 |
| A92 | 335 | 1020 | 3 |
| A93 | 132 | 276 | 2 |
| A94 | 438 | 869 | 2 |
| A95 | 10000 | 10000 | — |
| A96 | 1110 | 10000 | 9 |
| A99 | 1800 | 10000 | 5.6 |
| A100 | 283 | 1430 | 5.1 |
| A101 | 822 | 3840 | 4.7 |
| A102 | 10000 | 10000 | 1 |
| A103 | 7680 | 10000 | 1.3 |
| A116 | ≤2.5 | 4.1 | 1.9 |
| A117 | 6490 | 10000 | 1.5 |
| A118 | 3.7 | 6.6 | 1.8 |
| A122 | 31 | 160 | 5 |
| A123 | 41 | 201 | 5 |
| A128 | 8.9 | 34 | 3.8 |
| A129 | ≤2.5 | 3.5 | 2.4 |
| A131 | 5.7 | 10 | 1.8 |
| A133 | 265 | 797 | 3 |
| A138 | 5 | 24 | 4.8 |
| A139 | 5.5 | 18 | 3.2 |
| A140 | 11 | 27 | 2.5 |
| A141 | 10000 | 10000 | 1 |
| A142 | 10000 | 10000 | 1 |
| A143 | 3000 | 10000 | 3 |
| A144 | 32 | 90 | 2.5 |
| A145 | 36 | 72 | 2 |
| A146 | 219 | 178 | 1 |
| A147 | 13 | 15 | 1 |
| A148 | 18 | 17 | 1 |
| A149 | ≤2.5 | 3 | ≤1 |
| A150 | 23 | 50 | 2 |
| A151 | 1110 | | |
| A152 | 1060 | 4630 | 2 |
| A153 | 266 | 605 | |
| A154 | 3 | 3 | 1 |
| A155 | 6 | 19 | 3 |
| A156 | 54 | 248 | 5 |
| A157 | 67 | 64 | 1 |
| A158 | 24 | 16 | |
| A159 | 18 | 54 | 3 |
| A160 | 33 | 95 | 3 |
| A161 | 9 | 20 | 2 |
| A162 | 89 | 362 | 4 |
| A163 | 76 | 75 | 1 |
| A164 | 75 | 226 | 3 |
| A165 | 57 | 238 | 4 |
| A166 | 5 | 5 | 1 |
| A167 | 154 | 614 | 4 |
| A168 | | | |
| A169 | 20 | 62 | 3 |
| A170 | 4 | 7 | 2 |
| A171 | 16 | 25 | 2 |
| A172 | 5 | 11 | 2 |
| A173 | 52 | 244 | 5 |
| A174 | 7 | 33 | 4 |
| A175 | 36 | 129 | 4 |
| A176 | 7 | 13 | 2 |
| A177 | ≤2.5 | 3 | ≤1 |
| A178 | ≤2.5 | 10 | ≤4 |
| A179 | ≤2.5 | ≤2.5 | ≤1 |
| A180 | 4 | 6 | |
| A181 | ≤2.5 | ≤2.5 | ≤1 |
| A200 | 1340 | 2380 | 2 |

Triple-Negative Breast Cancer Cell Death Assay to Measure LD₅₀: The efficacy and selectivity of the disclosed compounds was evaluated in a TNBC cell line assay (MDA-MB-231) and/or human foreskin fibroblast 2088 cells, as was protein binding to human serum and microsomal stability of the disclosed compounds, as described herein.

Cells from a TNBC cell line (MDA-MB-231) (approximately 100,000 cells) and/or human foreskin fibroblast 2088 cells were seeded into each well of a 6-welled polystyrene treated tissue culture plate and were allowed to attach for 18 h before treatment with the test compound. Cells were then treated every 24 h with a fresh batch of media containing the test compound at one of the respective concentration(s) (Log₁₀ Molar [C]): −5.0, −5.5, −6.0, −6.5, −7.0, −7.5 so that a complete dose-response could be calculated.

After 48 h of treatment, adherent cells were fixed with a 4% paraformaldehyde solution and stained with Hoechst 33342 nuclear dye (5 μg/mL). Each well was imaged at 10× magnification on a Keyence BZ-X710 microscope and live cell counts were performed in triplicate for each condition using automated ImageJ computational software. LD₅₀ values represent the percentage of live cells after treatment with the test compound, which was calculated after normalization to the DMSO only treated control group.

Microsome Assay to Measure Stability: The stability of the compounds was evaluated using a liver cell microsome assay commercially available from Cyprotex (Watertown, MA) (https://www.cyprotex.com/admepk/in-vitro-metabolism/microsomal-stability).

The results of the LD₅₀ and microsome stability are summarized in Table 2.

TABLE 2
LD₅₀, Permeability and Microsome Stability

| | | Human | Mouse |
|---|---|---|---|
| | MDA LD₅₀ | Microsome | Microsome |
| Compound¹ | (nM) | Stability² | Stability² |
| A11 | 180 | 28.7 | 31.3 |
| A21 | 178 | 159.0 | 93.0 |
| A39 | 158 | 7.6 | 38.6 |
| A53 | 91 | 36 | 19 |

¹Fibroblast LD₅₀ >10,000 (nM) for each compound tested
²(μL/min/mg protein)

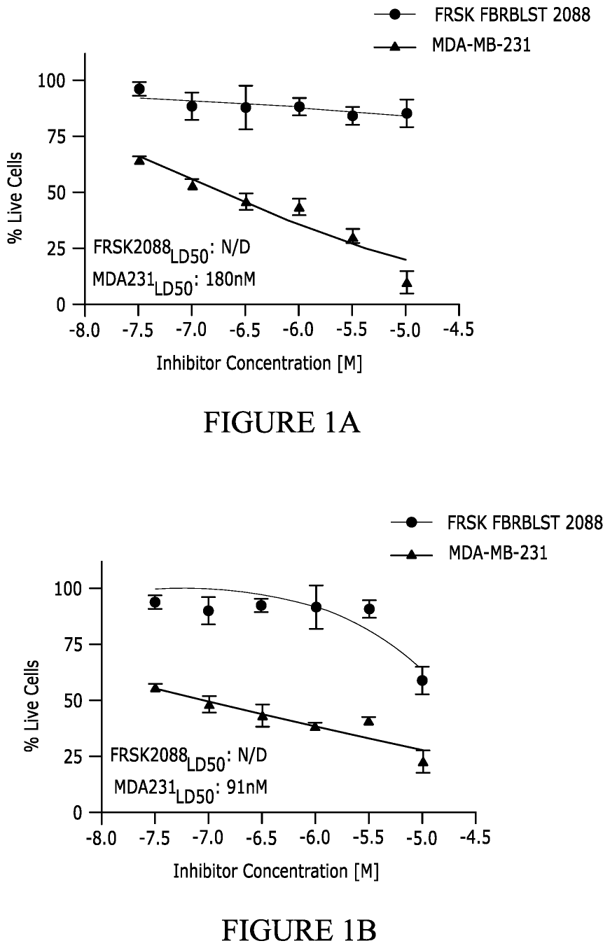

The results of the LD₅₀ studies are shown in FIGS. 1A (Compound A10) and 1B (Compound A47). Normal human fibroblast (circles) and MDA-MB-231 TNBC (triangles) cells were treated with various concentrations of test compounds and live cell counts were performed and plotted to determine a LD₅₀ for each compound. Live cell counts were normalized to the DMSO only treated control group. The only significant cell death observed for fibroblast cells was at 10,000 nM for Compound A47. In contrast, significant MDA-MB-231 TNBC cell death was observed for all compounds at concentrations below 500 nM.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein, is intended merely to better illuminate the invention and does not pose a limitation on the scope of the invention unless otherwise claimed. No language in the specification should be construed as indicating any non-claimed element as essential to the practice of the invention.

1. A compound, tautomer, or pharmaceutically acceptable salt thereof, having a structure of formula (I): 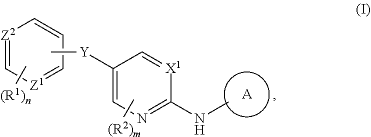
wherein:
X¹ is CH, CR², or N;
Y is selected from the group consisting of a bond, CR^{a}R^{b}, NR^{c}, C(O), O, S, SO₂, C(O)NH, and HNC(O);
each of Z¹ and Z² is independently CH, CR¹, or N;
each of R^{a} and R^{b} is independently H, C₁-C₆alkyl, hydroxy, or halo, or R^{a} and R^{b} taken together with the carbon atom which they are attached form a spiro C₃-C₆cycloalkyl;
R^{c} is H or C₁-C₆alkyl;
ring A comprises a C₆-C₁₀aryl, a C₃-C₆ cycloalkyl, or a 6-membered cycloheteroalkyl comprising 1, 2, or 3 ring heteroatoms independently selected from N, O, and S, wherein ring A is optionally substituted with 1-3 substituents independently selected from the group consisting of halo, hydroxy, —CN, C₁-C₆alkyl, C₁-C₆haloalkyl, C₃-C₆cycloalkyl, a spiro C₃-C₆ cycloalkyl, C₁-C₆alkoxy, C₁-C₆haloalkoxy, C₃-C₆cycloalkoxy, C₃-C₆cycloalkyl-C₁-C₆alkylene, C₆-C₁₀aryl, C₅-C₁₀cycloalkyl, 5-10 membered cycloheteroalkyl comprising 1, 2, or 3 ring heteroatoms independently selected from N, O, and S, NR′R″, and C(O)NR′R″;
n is 0, 1, or 2;
each R¹ is independently selected from the group consisting of halo, hydroxy, cyano, C₁-C₆alkyl, C₁-C₆haloalkyl, C₃-C₆cycloalkyl, C₁-C₆alkoxy, C₁-C₆haloalkoxy, C₂-4alkynylene-phenyl, C₃-C₆cycloalkoxy optionally substituted with C₁-C₆alkyl, C₅-C₆heteroaryl comprising 1, 2, or 3 ring heteroatoms independently selected from N, O, and S NR′R″, C(O)NR′R″, and 6-10 membered cycloheteroalkoxy comprising 1, 2, or 3 ring heteroatoms independently selected from N, O, and S, and the cycloalkyl, cycloalkoxy, phenyl, heteroaryl, and cycloheteroalkoxy ring is substituted with 0, 1, or 2 substituents independently selected from C₁₋₆alkyl, halo, C₁₋₆alkoxy, and C₃₋₆cycloalkyl; or
when two R¹ are ortho to each other, taken together with the atoms to which they are attached they form a fused 5 or 6 membered aromatic ring comprising 0-3 ring heteroatoms independently selected from N, O, and S, and is optionally substituted with 1-2 substituents independently selected from C₁-C₆alkyl and oxo;
m is 0, 1, or 2;
each R² is independently C₁-C₆ alkyl;
each R′ and R″ is independently selected from the group consisting of H, C₁-C₁₀alkyl, and C₃-C₆cycloalkyl; or taken together with the nitrogen to which they are attached form a 4-8 membered heterocycle including 0-2 additional ring heteroatoms independently selected from N, O, and S;
with the proviso that when X¹ is N, m is 0, Z¹ is N, Y is para to Z¹, Z² is CH, ring A is phenyl optionally substituted with NH₂ or CH₃, n is 0 or 2, and each R¹ is NH₂, then Y is not a bond.

2. The compound, tautomer, or salt of claim 1, wherein each of Z¹ and Z² is independently CH or N.

3. (canceled)

4. (canceled)

5. The compound, tautomer, or salt of claim 1, wherein Z¹ is N or NH and Z² is CH.

6. (canceled)

7. The compound, tautomer, or salt of claim 5, wherein the structure of formula (I) is a structure selected from one of formulae (IA)-(IG):

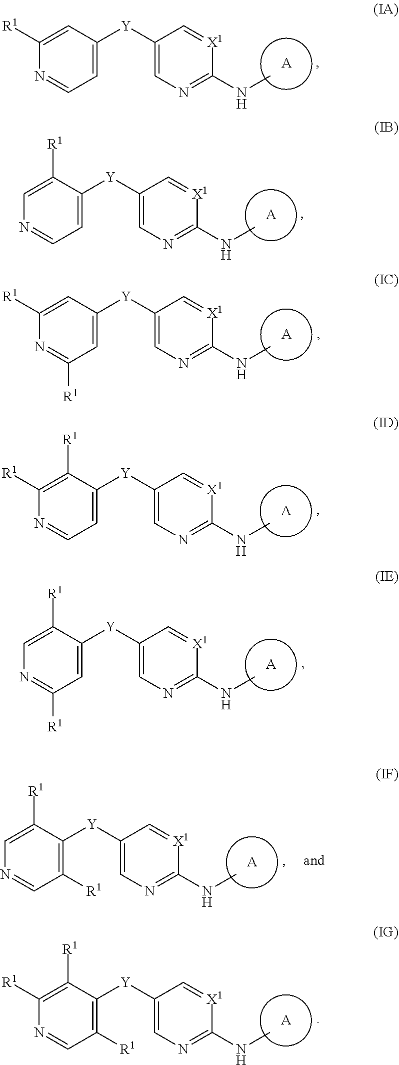

8. (canceled)

9. (canceled)

10. (canceled)

11. (canceled)

12. The compound, tautomer, or salt of claim 1, wherein each R1 is independently selected from the group consisting of halo, hydroxy, cyano, C₁-C₆alkyl, C₁-C₆haloalkyl, C₁-C₆cycloalkyl, C₁-C₆alkoxy, C₁-C₆haloalkoxy, C₃-C₆cycloalkoxy, NR′R″, C(O)NR′R″, and 6-10 membered cycloheteroalkoxy comprising 1, 2, or 3 ring heteroatoms independently selected from N, O, and S.

13. (canceled)

14. (canceled)

15. The compound, tautomer, or salt of claim 1, wherein at least one R¹ is hydrogen, cyclopentoxy, methyl, methoxy, isopropyl, trifluoromethyl, —C(O)NHMe, N-methylacetamido, ethyl, cyclohexoxy, piperidin-3-yl-O—, fluoro, chloro, trifluoromethoxy, cyclopropyl, cyclopentylNH—, cyano, oxo, cyclopentyl-NHC(O)—, —CH₂—NH—C(O)—, 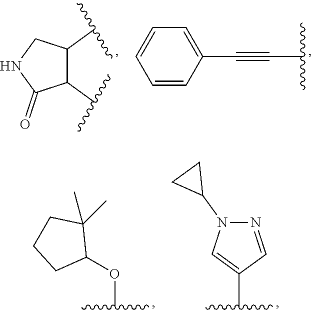
or amino, or two ortho R¹ together form

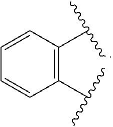 [SMILES: *c1ccccc1*]

16. (canceled)

17. (canceled)

18. (canceled)

19. (canceled)

20. The compound, tautomer, or salt of claim 1, wherein two R¹ are ortho to each other and taken together with the atoms to which they are attached form a 6 membered aryl, and the aryl is optionally substituted with methyl.

21. (canceled)

22. (canceled)

23. (canceled)

24. (canceled)

25. The compound, tautomer, or salt of claim 1, wherein ring A is optionally substituted with 1, 2, or 3 substituents selected from the group consisting of F, Cl, Br, I, hydroxy, NH₂, NHR′, methyl, ethyl, propyl, cyclopropyl, butyl, cyclobutyl, isobutyl, tert-butyl, pentyl, cyclopentyl, hexyl, cyclohexyl, methoxy, fluoromethoxy, difluoromethoxy, trifluoromethoxy, ethoxy, 1,1,2,2-tetrafluoroethoxy, perfluoroethoxy, propoxy, isopropoxy, cyclopropoxy, butoxy, cyclobutoxy, isobutoxy, tert-butoxy, pentoxy, cyclopentoxy, hexoxy, cyclohexoxy, cyclopropylmethyl, cyclobutylmethyl, piperazinyl, morpholinyl, 1-naphthyl, 2-naphthyl, tetrahydronapthyl, and isocromenyl.

26. The compound, tautomer, or salt of claim 1, wherein ring A is selected from the group consisting of phenyl, cyclohexyl, 4-piperidinyl, and tetrahydropyranyl, wherein ring A is optionally substituted.

27. (canceled)

28. The compound, tautomer, or salt of claim 1, wherein
(i)_ring A is cyclohexyl substituted at the 4-position with a substituent selected from the group consisting of methyl, methoxy, and isopropoxy; or
(ii) ring A is 4-piperidinyl substituted on ring N with methyl or isobutyl; or
(iii) ring A is phenyl substituted at the 2-position with a substituent selected from the group consisting of F, Cl, Br, I, C₁-C₆alkoxy, hydroxy, NH₂, and NHR′; or
(iv) ring A is phenyl substituted at the 3-position or 4-position with a substituent selected from the group consisting of F, Cl, Br, I, C₁-C₆alkyl, C₁-C₆alkoxy, C₁-C₆-haloalkyl, C₃-C₆cycloalkyl, C₃-C₆cycloalkoxy, C₃-C₆cycloalkyl-C₁-C₆alkylene, C₆-C₁₀aryl, C₆-C₁₀cycloalkyl, and 6-10 membered heterocycloalkyl comprising 1, 2, or 3 ring heteroatoms independently selected from N, O, and S.

29. (canceled)

30. (canceled)

31. (canceled)

32. (canceled)

33. (canceled)

34. (canceled)

35. (canceled)

36. (canceled)

37. (canceled)

38. The compound, tautomer, or salt of claim 1, wherein X¹ is CH or N.

39. The compound, tautomer, or salt of claim 1, wherein X¹ is N and m is 0; or X¹ is N, M is 1 and R² is methyl.

40. (canceled)

41. (canceled)

42. The compound, tautomer, or salt of claim 1, wherein Y is NH.

43. (canceled)

44. (canceled)

45. (canceled)

46. (canceled)

47. The compound, tautomer, or salt of claim 1, wherein each of R^{a} and R^{b} is independently H or C₁-C₆alkyl.

48. The compound, tautomer, or salt of claim 1 having a structure as recited in Table A.

49. (canceled)

50. A method of inhibiting cyclin dependent kinase 19 (CDK19) comprising contacting CDK19 with the compound, tautomer, or salt of claim 1 in an amount effective to inhibit CDK19.

51. The method of claim 50, wherein the compound inhibits CDK19 selectively over cyclin dependent kinase 8 (CDK8).

52. (canceled)

53. (canceled)

54. (canceled)

55. (canceled)

56. (canceled)

57. A method of treating cancer in a patient comprising administering to the patient a therapeutically effective amount of the compound, tautomer, or salt of claim 1.

58. The method of claim 57, wherein the cancer is breast cancer, prostate cancer, cancer of the gastrointestinal tract (e.g., colorectal cancer), bladder cancer, sarcoma, cervical cancer, esophageal adenocarcinoma, acute myeloid leukemia, melanoma, glioma, or ovarian cancer.

59. (canceled)

60. (canceled)

61. (canceled)

62. (canceled)

63. (canceled)
